(19) United States
(12) Patent Application Publication
(10) Pub. No.: US 2023/0123762 A1
(43) Pub. Date: Apr. 20, 2023

(54) BIO-THERAPEUTICS FOR DETECTION, DIAGNOSIS AND TREATMENT OF DISEASES ASSOCIATED WITH MUCOSAL BLEEDING

(71) Applicant: TENZA, INC., Cambridge, MA (US)

(72) Inventor: Anik DEBNATH, Boston, MA (US)

(21) Appl. No.: 17/904,038

(22) PCT Filed: Feb. 11, 2021
(86) PCT No.: PCT/US2021/017683

§ 371 Date: Aug. 11, 2022

Related U.S. Application Data
(60) Provisional application No. 62/975,186, filed on Feb. 11, 2020.

(51) Int. Cl. C12N 15/74; C12Q 1/02; A61P 1/00; A61P 7/04; A61K 35/747

(52) U.S. Cl. CPC C12N 15/746; C12Q 1/02; A61P 1/00; A61P 7/04; A61K 35/747; C12N 2830/005; A61K 2035/115

(57) ABSTRACT

Provided herein are embodiments directed to heme-responsive promoter sequences, heme-responsive repressor systems which may be used as probiotic bacteria and as bio-therapeutics, such probiotic bacteria and bio-therapeutics are described in methods of detecting bleeding in a mucosal environment, diagnosing in vivo bleeding in a mucosal environment, and treating in vivo bleeding in a mucosal environment.

### CROSS-REFERENCE TO RELATED APPLICATION

The present application is a 35 U.S.C. § 371 National Stage application of International Application Number PCT/US2021/017683, filed Feb. 11, 2021, which claims priority to U.S. Provisional Application No. 62/975,186, filed Feb. 11, 2020, each of which is hereby incorporated by reference in their entirety.

### SEQUENCE LISTING

The instant application contains a Sequence Listing which has been submitted electronically in ASCII format and is hereby incorporated by reference in its entirety. Said ASCII copy, created on Aug. 11, 2022, is named 257723.000301_sequence_listing_ST25 and is 172,347 bytes in size.

### FIELD

The present disclosure relates generally to methods of regulating gene expression in a cell and more particularly to regulating gene expression in the presence of heme.

### BACKGROUND

Gastrointestinal (GI) bleeding is a major cause of morbidity and mortality. Underlying causes of gastrointestinal bleeding include angiodyslpasia, tumors, gastric ulcers, colitis, diverticular disease, esophageal varices, Crohn's disease, irritable bowel syndrome, hemorrhoids and peptic ulcers. Typically, several tests, such as stool and blood analysis, gastric lavage, endoscopy, enteroscopy, colonoscopy, flexible sigmoidoscopy, and imaging tests, are used to determine the cause of GI bleeding. These tests suffer from drawbacks, are inconvenient and contribute to rising health care costs. Therefore, there exists an ongoing need for quick, effective and convenient tools and methods for detecting and diagnosing GI bleeding.

### SUMMARY

In one aspect, the present disclosure provides a heme-responsive promoter comprising a nucleic acid sequence that is at least 70% homologous to SEQ ID NO:104, which may be fused to or introduced into any constitutive promoter.

In another aspect, the present disclosure provides a heme-responsive repressor system comprising: a first nucleic acid comprising a first constitutive promoter of a lactic acid bacteria and one or more hrtO repressor binding sites; and a second nucleic acid comprising a second constitutive promoter operably linked to a gene encoding an HrtR protein, wherein the HrtR protein is capable of binding to heme and the hrtO repressor binding sites, wherein when bound to heme, the HrtR protein is not capable of binding to the hrtO repressor binding site and wherein when not bound to heme, the HrtR protein is capable of binding to the hrtO repressor binding site.

In any embodiment, the HrtR protein may have an amino acid sequence that is at least 90% identical to any one of SEQ ID NOs: 1-100.

In yet another aspect, the present disclosure provides a heme-responsive repressor system comprising: a first nucleic acid comprising a heme-responsive promoter comprising a first constitutive promoter fused to an amino acid sequence that is at least 90% homologous to SEQ ID NO:104; and a second nucleic acid comprising a second constitutive promoter operably linked to a gene encoding an HrtR protein that binds to the hrtO repressor binding site in SEQ ID NO:104, wherein when bound to heme, the HrtR protein is not capable of binding to the hrtO repressor binding site and wherein when not bound to heme, the HrtR protein is capable of binding to the hrtO repressor binding site.

In yet another aspect, the present disclosure provides an engineered bacteria, for example a lactic acid bacteria, comprising a bacteria genetically modified with the heme-responsive repressor system described above.

The engineered bacteria may be incorporated, for example, into a pharmaceutical composition administrable to a subject for use detecting bleeding in a mucosal environment of the subject.

Therefore in another aspect, the present disclosure provides a method of detecting bleeding in a mucosal environment of a subject comprising: administering to a subject in need thereof a composition comprising an engineered bacteria genetically modified with a first nucleic acid comprising a heme-responsive promoter comprising one or more hrtO repressor binding sites that binds a heme-sensitive transcriptional repressor HrtR protein; and a second nucleic acid operably linked to a gene encoding the HrtR protein; and detecting the presence of the reporter protein in the subject.

The engineered bacteria may also be incorporated into a pharmaceutical composition for delivery of a therapeutic protein or peptide to a subject. Therefore, in yet another aspect, the present disclosure provides a method of prescriptively treating gastric bleeding disorder in a mucosal environment of a subject comprising: administering to a subject in need thereof a composition comprising an engineered bacteria genetically modified with a first nucleic acid comprising a heme-responsive promoter comprising one or more hrtO repressor binding sites, operably linked to a therapeutic protein, that binds a heme-sensitive transcriptional repressor HrtR protein; and a second nucleic acid operably linked to a gene encoding the HrtR protein; and produce a therapeutic product to subject's gastrointestinal tissues exhibiting bleeding, in scale with the amount of heme present in the mucosal environment.

### BRIEF DESCRIPTION OF THE DRAWINGS

The foregoing and other features of the disclosure will become more apparent from the following detailed description of several embodiments, which proceeds with reference to the accompanying figures.

FIG. 1 is a schematic representation showing the location and transcriptional regulatory function of the hrtO repressor binding site from Lactobacillus lactis within the 5′ UTR region of transcriptional start site (+1) of the slpA promoter from Lactobacillus acidophilus NCFM

FIG. 2 provides SEQ ID NO:102, the nucleic acid sequence of the heme-responsive version of the slpA promoter from Lactobacillus acidophilus NCFM modified with two hrtO repressor binding sites.

FIG. 3 provides the 5′ UTR of the slpA promoter from Lactobacillus acidophilus NCFM

FIG. 4 provides SEQ ID NO:104, the 5′ UTR of the slpA promoter from Lactobacillus acidophilus NCFM modified with two hrtO repressor binding sites from Lactobacillus lactis.

FIG. 5 provides SEQ ID NO:105, the nucleic acid sequence of the heme-responsive promoter clpC from Lactobacillus fermentum BR11, modified with the 5′ UTR of the slpA promoter from Lactobacillus acidophilus NCFM.

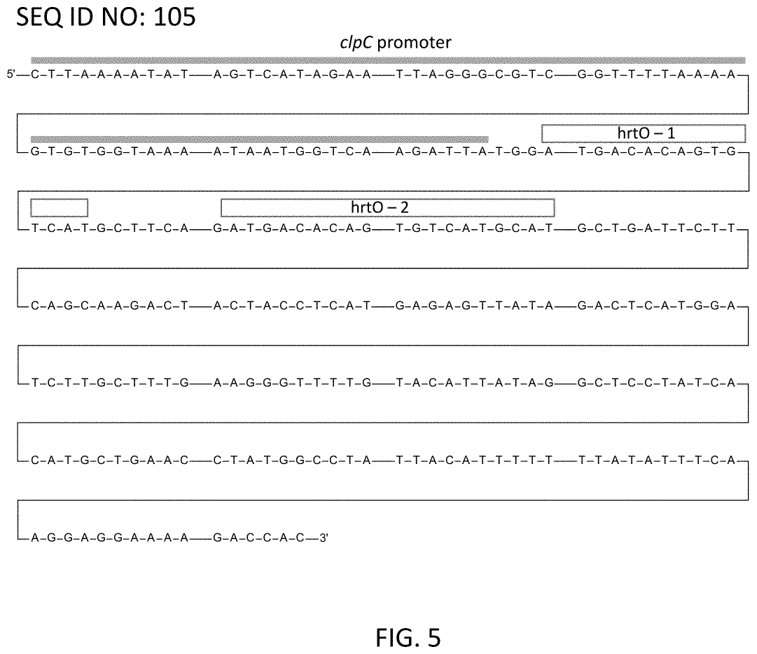

FIG. 6 depicts the in vitro response of genetically modified Lactobacillus rhamnosus GG to heme presence over a 12 hour time period.

FIG. 7 provides the data from FIG. 6, normalized to bacterial growth.

FIG. 8 depicts heme-tolerance by Lactobacillus rhamnosus GG over a range of heme concentrations.

FIG. 9 provides an image of several mouse colons after administration of Lactobacillus rhamnosus modified with a heme-responsive repressor system as described herein to the mice, wherein the heme-responsive promoter is operably linked to a reporter gene encoding the expression of a fluorescent protein. Mice were either provided normal drinking water (left) or % DSS-treated drinking water, which induced bleeding (right).

### DETAILED DESCRIPTION

The present disclosure presents a solution to the aforementioned challenges of detecting and diagnosing GI bleeding by providing engineered bacteria that are programmed to express a reporter protein in response to heme in an in vivo mucosal environment, such as the GI tract. Gene expression is proportional to the amount of heme (blood) stimulus in the mucosal environment. The disclosed bacteria may also be engineered to deliver a therapeutic agent in the presence of heme at a dose scaled to the severity of the sensed bleeding.

### Definitions

The following explanations of terms and methods are provided to better describe the present disclosure and to guide those of ordinary skill in the art in the practice of the present disclosure.

As used herein, “comprising” means “including” and the singular forms “a” or “an” or “the” include plural references unless the context clearly dictates otherwise. For example, reference to “comprising a therapeutic agent” includes one or a plurality of such therapeutic agents. The term “or” refers to a single element of stated alternative elements or a combination of two or more elements, unless the context clearly indicates otherwise. For example, the phrase “A or B” refers to A, B, or a combination of both A and B. Furthermore, the various elements, features and steps discussed herein, as well as other known equivalents for each such element, feature or step, can be mixed and matched by one of ordinary skill in this art to perform methods in accordance with principles described herein. Among the various elements, features, and steps some will be specifically included and others specifically excluded in particular examples.

Unless otherwise indicated, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. All references cited herein are incorporated by reference in their entirety.

In some examples, the numbers expressing quantities of ingredients, properties such as molecular weight, reaction conditions, and so forth, used to describe and claim certain embodiments are to be understood as being modified in some instances by the term “about” or “approximately.” For example, “about” or “approximately” can indicate +/−5% variation of the value it describes. Accordingly, in some embodiments, the numerical parameters set forth herein are approximations that can vary depending upon the desired properties for a particular embodiment. Notwithstanding that the numerical ranges and parameters setting forth the broad scope of some examples are approximations, the numerical values set forth in the specific examples are reported as precisely as practicable. The recitation of ranges of values herein is merely intended to serve as a shorthand method of referring individually to each separate value falling within the range.

To facilitate review of the various embodiments of this disclosure, the following explanations of specific terms are provided:

Administer: To provide or give a subject a composition by an effective route. Exemplary routes of application include, but are not limited to, oral, enteral, parenteral and topical routes.

Antibiotic: A chemical substance capable of treating bacterial infections by inhibiting the growth of, or by destroying existing colonies of bacteria and other microorganisms.

Anti-inflammatory agent: An active agent that reduces inflammation and swelling.

Antioxidant: An active agent that inhibits oxidation or reactions promoted by oxygen or peroxides.

Cancer: A condition characterized by unregulated cell growth. Examples of cancer include, but are not limited to, squamous cell cancer, small-cell lung cancer, non-small cell lung cancer, lung adenocarcinoma, lung squamous cell carcinoma, gastrointestinal cancer, Hodgkin's and non-Hodgkin's lymphoma, pancreatic cancer, glioblastoma, cervical cancer, colon cancer, colorectal cancer, endometrial or uterine carcinoma, kidney cancer such as renal cell carcinoma and Wilms' tumors, basal cell carcinoma, melanoma, prostate cancer, and esophageal cancer.

Contacting: Placement in direct physical association; includes both in solid and liquid form.

Drug or Active Agent: A chemical substance or compound that induces a desired pharmacological or physiological effect, and includes agents that are therapeutically effective, prophylactically effective, or cosmeceutically effective. The terms also encompass pharmaceutically acceptable, pharmacologically active derivatives and analogs of those active agents specifically mentioned herein, including, but not limited to, salts, esters, amides, prodrugs, active metabolites, inclusion complexes, analogs, and the like. Suitable active agents may include, but are not limited to, alcohol deterrents; amino acids; ammonia detoxicants; anabolic agents; analeptic agents; analgesic agents; androgenic agents; anesthetic agents; anorectic compounds; anorexic agents; antagonists; anti-allergic agents; anti-amebic agents; anti-anemic agents; anti-anginal agents; anti-anxiety agents; anti-arthritic agents; anti-atherosclerotic agents; antibacterial agents; anti-cancer agents, including antineoplastic drugs, and anti-cancer supplementary potentiating agents; anticholinergics; anticholelithogenic agents; anticoagulants; anti-coccidal agents; anti-convulsants; anti-depressants; anti-diabetic agents; anti-diarrheals; anti-diuretics; antidotes; anti-dyskinetics agents; anti-emetic agents; antiepileptic agents; anti-estrogen agents; anti-fibrinolytic agents; anti-fungal agents; anti-glaucoma agents; anti-hemophilic agents; anti-hemorrhagic agents; antihistamines; anti-hyperlipidemic agents; anti-hyperlipoproteinemic agents; antihypertensive agents; anti-hypotensives; anti-infective agents such as antibiotics and antiviral agents; anti-inflammatory agents, both steroidal and non-steroidal; anti-keratinizing agents; anti-malarial agents; antimicrobial agents; anti-migraine agents; anti-mitotic agents; anti-mycotic agents; antinauseants; antineoplastic agents; anti-neutropenic agents; anti-obsessional agents; anti-parasitic agents; antiparkinsonism drugs; anti-pneumocystic agents; anti-proliferative agents; anti-prostatic hypertrophy drugs; anti-protozoal agents; antipruritics; anti-psoriatic agents; antipsychotics; antipyretics; antispasmodics; antirheumatic agents; anti-schistosomal agents; anti-seborrheic agents; anti-spasmodic agents; anti-thrombotic agents; anti-tubercular agents; antitussive agents; anti-ulcerative agents; anti-urolithic agents; antiviral agents; GERD medications, anxiolytics; appetite suppressants; attention deficit disorder (ADD) and attention deficit hyperactivity disorder (ADHD) drugs; bacteriostatic and bactericidal agents; benign prostatic hyperplasia therapy agents; blood glucose regulators; bone resorption inhibitors; bronchodilators; carbonic anhydrase inhibitors; cardiovascular preparations including anti-anginal agents, anti-arrhythmic agents, beta-blockers, calcium channel blockers, cardiac depressants, cardiovascular agents, cardioprotectants, and cardiotonic agents; central nervous system (CNS) agents; central nervous system stimulants; choleretic agents; cholinergic agents; cholinergic agonists; cholinesterase deactivators; coccidiostat agents; cognition adjuvants and cognition enhancers; cough and cold preparations, including decongestants; depressants; diagnostic aids; diuretics; dopaminergic agents; ectoparasiticides; emetic agents; enzymes which inhibit the formation of plaque, calculus or dental caries; enzyme inhibitors; estrogens; fibrinolytic agents; fluoride anticavity/antidecay agents; free oxygen radical scavengers; gastrointestinal motility agents; genetic materials; glucocorticoids; gonad-stimulating principles; hemostatic agents; herbal remedies; histamine H2 receptor antagonists; hormones; hormonolytics; hypnotics; hypocholesterolemic agents; hypoglycemic agents; hypolipidemic agents; hypotensive agents; immunizing agents; immunomodulators; immunoregulators; immunostimulants; immunosuppressants; impotence therapy adjuncts; inhibitors; keratolytic agents; leukotriene inhibitors; liver disorder treatments; metal chelators such as ethylenediaminetetraacetic acid, tetrasodium salt; mitotic inhibitors; mood regulators; mucolytics; mucosal protective agents; muscle relaxants; mydriatic agents; narcotic antagonists; neuroleptic agents; neuromuscular blocking agents; neuroprotective agents; nicotine; NMDA antagonists; non-hormonal sterol derivatives; nutritional agents, such as vitamins, essential amino acids and fatty acids; ophthalmic drugs such as antiglaucoma agents; oxytocic agents; pain relieving agents; parasympatholytics; peptide drugs; plasminogen activators; platelet activating factor antagonists; platelet aggregation inhibitors; post-stroke and post-head trauma treatments; potentiators; progestins; prostaglandins; prostate growth inhibitors; proteolytic enzymes as wound cleansing agents; prothyrotropin agents; psychostimulants; psychotropic agents; radioactive agents; regulators; relaxants; repartitioning agents; scabicides; sclerosing agents; sedatives; sedative-hypnotic agents; selective adenosine A1 antagonists; serotonin antagonists; serotonin inhibitors; serotonin receptor antagonists; steroids, including progestogens, estrogens, corticosteroids, androgens and anabolic agents; smoking cessation agents; stimulants; suppressants; sympathomimetics; synergists; thyroid hormones; thyroid inhibitors; thyromimetic agents; tranquilizers; tooth desensitizing agents; tooth whitening agents such as peroxides, metal chlorites, perborates, percarbonates, peroxyacids, and combinations thereof; unstable angina agents; uricosuric agents; vasoconstrictors; vasodilators including general coronary, peripheral and cerebral; vulnerary agents; wound healing agents; xanthine oxidase inhibitors; and the like.

Effective amount: The amount of an active agent (alone or with one or more other active agents) sufficient to induce a desired response, such as to prevent, treat, reduce and/or ameliorate a condition. Effective amounts of an active agent, alone or with one or more other active agents, can be determined in many different ways, such as assaying for a reduction in of one or more signs or symptoms associated with the condition in the subject or measuring the level of one or more molecules associated with the condition to be treated.

Expression: the process by which a protein or peptide is produced from DNA. The process involves the transcription of a gene into mRNA and the translation of the mRNA into a protein or peptide.

Gene: A nucleic acid sequence that undergoes transcription as the result of promoter activity. A gene may code for a particular protein or peptide.

Mucosa: A membrane that lines various cavities in the body and covers the surface of internal organs. It consists of one or more layers of epithelial cells overlying a layer of loose connective tissue. The mucosa is mostly of endodermal origin and is continuous with the skin at various body openings such as the eyes, ears, inside the nose, inside the mouth, lip, vagina, the urethral opening and the anus. Some mucous membranes secrete mucus, a thick protective fluid. The function of the membrane is to stop pathogens and dirt from entering the body and to prevent bodily tissues from becoming dehydrated.

Mucosal Administration: Administration through the mouth, nose, vagina, eyes and ears of a subject.

Operably linked: describes genetic elements that are joined in such a manner that enables them to carry out their normal functions. For example, a gene is operably linked to a promotor when its transcription is under the control of the promotor and such transcription produces the protein normally encoded by the gene.

Oral administration: Delivery of an active agent through the mouth.

pH Adjuster or Modifier: A molecule or buffer used to achieve desired pH control in a formulation. Exemplary pH modifiers include acids (e.g., acetic acid, adipic acid, carbonic acid, citric acid, fumaric acid, phosphoric acid, sorbic acid, succinic acid, tartaric acid, basic pH modifiers (e.g., magnesium oxide, tribasic potassium phosphate), and pharmaceutically acceptable salts thereof.

Probiotics: Live microorganisms that, when administered in adequate amounts, confer a health benefit on the host. One example is a lactic acid bacteria. Examples of lactic acid bacterial cells that may be used in any embodiment or aspect disclosed herein include bacterial cells within the order Lactobacillales, further from the genus Lactobacillus, Leuconostoc, Pediococcus, Lactococcus, Streptococcus, Carnobacterium, Enterococcus, Oenococcus, Tetragenococcus, Vagococcus, or Weisella. For example, suitable bacterial cells include bacterial cells from the species Lactobacillus acetotolerans, Lactobacillus acidifarinae, Lactobacillus acidipiscis, Lactobacillus acidophilus, Lactobacillus agilis, Lactobacillus algidus, Lactobacillus alimentarius, Lactobacillus alvei, Lactobacillus alvi, Lactobacillus amylolyticus, Lactobacillus amylophilus, Lactobacillus amylotrophicus, Lactobacillus amylovorus, Lactobacillus animalis, Lactobacillus animata, Lactobacillus antri, Lactobacillus apinorum, Lactobacillus apis, Lactobacillus apodemi, Lactobacillus aquaticus, Lactobacillus aviarius, Lactobacillus backii, Lactobacillus bifermentans, Lactobacillus bombi, Lactobacillus bombicola, Lactobacillus brantae, Lactobacillus brevis, Lactobacillus brevisimilis, Lactobacillus buchneri, Lactobacillus cacaonum, Lactobacillus camelliae, Lactobacillus capillatus, Lactobacillus casei, Lactobacillus paracasei, Lactobacillus zeae, Lactobacillus catenefornis, Lactobacillus ceti, Lactobacillus coleohominis, Lactobacillus colini, Lactobacillus collinoides, Lactobacillus composti, Lactobacillus concavus, Lactobacillus coryniformis, Lactobacillus crispatus, Lactobacillus crustorum, Lactobacillus curieae, Lactobacillus curvatus, Lactobacillus delbrueckii, Lactobacillus dextrinicus, Lactobacillus diolivorans, Lactobacillus equi, Lactobacillus equicursoris, Lactobacillus equigenerosi, Lactobacillus fabifermentans, Lactobacillus faecis, Lactobacillus faeni, Lactobacillus farciminis, Lactobacillus farraginis, Lactobacillus fermentum, Lactobacillus floricola, Lactobacillus florum, Lactobacillus formosensis, Lactobacillus fornicalis, Lactobacillus fructivorans, Lactobacillus frumenti, Lactobacillus fuchuensis, Lactobacillus furfuricola, Lactobacillus futsaii, Lactobacillus gallinarum, Lactobacillus gasseri, Lactobacillus gastricus, Lactobacillus ghanensis, Lactobacillus gigeriorum, Lactobacillus ginsenosidimutans, Lactobacillus gorillae, Lactobacillus graminis, Lactobacillus guizhouensis, Lactobacillus halophilus, Lactobacillus hammesii, Lactobacillus hamsteri, Lactobacillus harbinensis, Lactobacillus hayakitensis, Lactobacillus heilongjiangensis, Lactobacillus helsingborgensis, Lactobacillus helveticus, Lactobacillus herbarum, Lactobacillus heterohiochii, Lactobacillus hilgardii, Lactobacillus hokkaidonensis, Lactobacillus hominis, Lactobacillus homohiochii, Lactobacillus hordei, Lactobacillus iatae, Lactobacillus iners, Lactobacillus ingluviei, Lactobacillus insectis, Lactobacillus insicii, Lactobacillus intermedius, Lactobacillus intestinalis, Lactobacillus iwatensis, Lactobacillus ixorae, Lactobacillus japonicus, Lactobacillus jensenii, Lactobacillus johnsonii, Lactobacillus kalixensis, Lactobacillus kefiranofaciens, Lactobacillus kefiri, Lactobacillus kimbladii, Lactobacillus kimchicus, Lactobacillus kimchiensis, Lactobacillus kisonensis, Lactobacillus kitasatonis, Lactobacillus koreensis, Lactobacillus kullabergensis, Lactobacillus kunkeei, Lactobacillus larvae, Lactobacillus leichmannii, Lactobacillus letivazi, Lactobacillus lindneri, Lactobacillus malefermentans, Lactobacillus mall, Lactobacillus manihotivorans, Lactobacillus mellifer, Lactobacillus mellis, Lactobacillus melliventris, Lactobacillus micheneri, Lactobacillus mindensis, Lactobacillus mixtipabuli, Lactobacillus mobilis, Lactobacillus modestisalitolerans, Lactobacillus mucosae, Lactobacillus mudanjiangensis, Lactobacillus murinus, Lactobacillus nagelii, Lactobacillus namurensis, Lactobacillus nantensis, Lactobacillus nasuensis, Lactobacillus nenjiangensis, Lactobacillus nodensis, Lactobacillus odoratitofui, Lactobacillus oeni, Lactobacillus oligofermentans, Lactobacillus ori, Lactobacillus oryzae, Lactobacillus otakiensis, Lactobacillus ozensis, Lactobacillus panis, Lactobacillus pantheris, Lactobacillus parabrevis, Lactobacillus parabuchneri, Lactobacillus paracollinoides, Lactobacillus parafarraginis, Lactobacillus parakefiri, Lactobacillus paralimentarius, Lactobacillus paraplantarum, Lactobacillus pasteurii, Lactobacillus paucivorans, Lactobacillus pentosus, Lactobacillus perolens, Lactobacillus plajomi, Lactobacillus plantarum, Lactobacillus pobuzihii, Lactobacillus pontis, Lactobacillus porcinae, Lactobacillus psittaci, Lactobacillus rapi, Lactobacillus rennanquilfy, Lactobacillus rennini, Lactobacillus reuteri, Lactobacillus rhamnosus, Lactobacillus rodentium, Lactobacillus rogosae, Lactobacillus rossiae, Lactobacillus ruminis, Lactobacillus saerimneri, Lactobacillus sakei, Lactobacillus salivarius, Lactobacillus sanfranciscensis, Lactobacillus saniviri, Lactobacillus satsumensis, Lactobacillus secaliphilus, Lactobacillus selangorensis, Lactobacillus senioris, Lactobacillus senmaizukei, Lactobacillus sharpeae, Lactobacillus shenzhenensis, Lactobacillus sicerae, Lactobacillus silagei, Lactobacillus siliginis, Lactobacillus similis, Lactobacillus songhuajiangensis, Lactobacillus spicheri, Lactobacillus sucicola, Lactobacillus suebicus, Lactobacillus sunkii, Lactobacillus taiwanensis, Lactobacillus thailandensis, Lactobacillus tucceti, Lactobacillus ultunensis, Lactobacillus uvarum, Lactobacillus vaccinostercus, Lactobacillus vaginalis, Lactobacillus vermiforme, Lactobacillus vespulae, Lactobacillus vini, Lactobacillus wasatchensis, Lactobacillus xiangfangensis, Lactobacillus yonginensis, or Lactobacillus zymae.

Promoter: a DNA sequence that initiates transcription of a gene. Promoters are typically found 5′ to the gene and located proximal to the start codon. A promoter may be modified to include one or more transcriptional repressor protein binding sites, as is disclosed herein, to regulate transcription in response to an inducing agent, such as heme.

Recombinant: describes a nucleic acid, protein, or peptide that is formed by experimentally recombining nucleic acid sequences and sequence elements. A recombinant host would be any host (e.g., bacteria) receiving a recombinant nucleic acid and the term “recombinant protein” refers to protein produced by such a host.

Repressor or transcriptional repressor protein: a molecule capable of inhibiting expression of a particular nucleic acid sequence, such as a gene, from a promoter. In effect, the molecule “represses” the expression of the gene from its promoter.

A repressor system includes DNA comprising a promoter modified with a transcriptional repressor protein binding site and its corresponding transcriptional repressor protein, which, when present, binds to the transcriptional repressor binding site. Binding of the an agent to the transcriptional repressor protein may induce conformational change to the repressor protein which, in turn, may affect binding to the transcriptional repressor protein binding site, thereby creating an agent-responsive repressor system. Such a repressor system can be used to inhibit transcription of an operably linked gene. While an agent-responsive promotor may generally be manufactured using any constitutive promoter sequence, logistically, construction of a viable sequence is not straightforward. For example, International Patent Application Publication No. WO2018183685 discloses a tetracycline-responsive promoter system whereby binding of the transcriptional repressor protein tetR to the repressor binding site inhibits downstream transcription. Binding of tetR to the tetO repressor binding site utilizes distinct binding mechanisms that are highly dependent on the structure of both tetR and tetO, both in relation to each other, but also in relation to the promoter sequence.

Sequence: A linear arrangement of amino acids or nucleotides. Each amino acid or nucleotide is connected via a chemical bond to an adjacent amino acid or nucleotide by a peptide bond or phosphodiester bond, respectively. It is to be understood a particular nucleic acid or amino acid sequence may be disclosed for a particular embodiments of the present disclosure, however, deviations from a recited sequence are permitted, for example, sequences having at least 70%, 80%, 85% homology, at least 90% homology, at least 95% homology, at least 97% homology, at least 98% homology or at least 99% homology to the particular sequence disclosed herein may be suitable.

Subject: A living multi-cellular vertebrate organism, a category that includes human and non-human mammals, as well as birds (such as chickens and turkeys), fish, and reptiles. Exemplary subjects include mammals, such as human and non-human primates, rats, mice, dogs, cats, rabbits, cows, pigs, goats, horses, and the like.

Surface or Body Surface: A surface located on the human body or within a body orifice. Thus, a “body surface” includes, by way of example, skin, teeth, mucosal tissue, including the interior surface of body cavities that have a mucosal lining.

Topical administration: Delivery of an active agent to a body surface, such as, the skin or mucosa, as in, for example, topical drug administration in the prevention or treatment of various skin disorders.

Transfection: Delivery of a vector comprising one or more nucleic acids to a bacteria, e.g., through a plasmid.

Ulcer: A break in the skin or mucous membrane with loss of surface tissue and the disintegration and necrosis of epithelial tissue. A mucosal ulcer specifically occurs on a mucous membrane.

Vector (or Expression Vector): a nucleic acid capable of transporting another nucleic acid linked thereto. Vectors and plasmids are common and commercially available from companies such as Invitrogen Corp. (Carlsbad, Calif.), Stratagene (La Jolla, Calif.), New England Biolabs, Inc. (Beverly, Mass.) and Addgene (Cambridge, Mass.). Any vector may be used to deliver a nucleic acid to bacterial cells in any embodiment disclosed herein.

For example, one type of vector is a “plasmid,” which refers to a circular double stranded DNA loop into which additional DNA segments can be ligated. Vectors include, but are not limited to, nucleic acid molecules that are single-stranded, double stranded, or partially double-stranded; nucleic acid molecules that comprise one or more free ends, no free ends (e.g., circular); nucleic acid molecules that comprise DNA, RNA, or both; and other varieties of polynucleotides known in the art. By way of example, but not of limitation, a vector can be a single copy or multi-copy vector, including, but not limited to, a BAC (bacterial artificial chromosome), a fosmid, a cosmid, a plasmid, a suicide plasmid, a shuttle vector, a P1 vector, an episome, or YAC (yeast artificial chromosome) or any other suitable vector.

Another type of vector is a viral vector, wherein virally-derived DNA or RNA sequences are present in the vector for packaging into a virus (e.g., retroviruses, lentiviruses, replication defective retroviruses, adenoviruses, replication defective adenoviruses, and adeno-associated viruses). Viral vectors also include polynucleotides carried by a virus for transfection into a host cell. Viral vectors include those where additional DNA segments can be ligated into the viral genome, a bacteriophage or viral genome, or any other suitable vector. The host cells can be any cells in which the vector is able to replicate.

Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” Common expression vectors of utility in recombinant DNA techniques are often in the form of plasmids. In the present specification, “plasmid” and “vector” can be used interchangeably. However, the methods disclosed herein may utilize other forms of expression vectors, such as viral vectors (e.g., replication defective retroviruses, adenoviruses and adeno-associated viruses), which serve equivalent functions.

Heme-Responsive Promoters & Repressor Systems

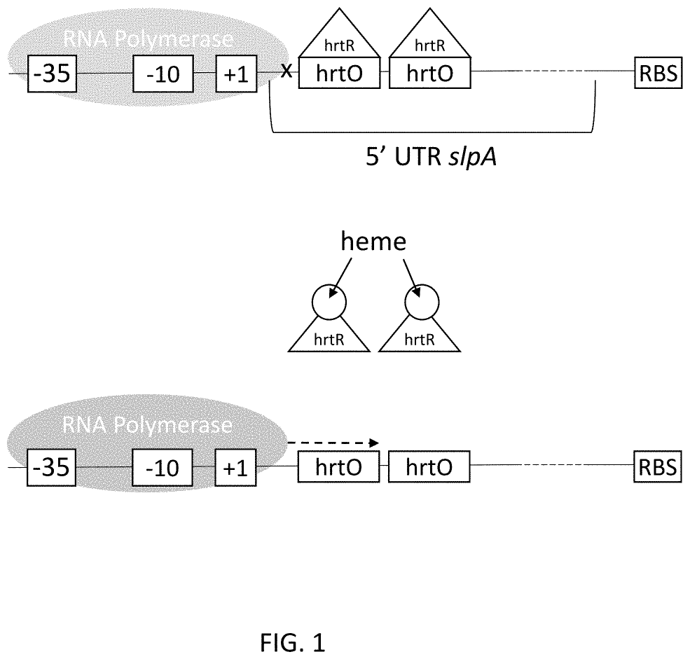

The present disclosure, in various aspects and embodiments, provides heme-responsive promoters comprising a constitutive promoter (e.g., any functional promoter) modified to include one or more hrtO repressor binding sites. When a repressor binding protein HrtR is bound to the repressor binding site, hrtO, transcription of operably linked downstream genes is inhibited. When HrtR is not bound to hrtO, transcription proceeds. The binding of HrtR to hrtO is controlled by a configuration shift of HrtR in response to heme binding. When heme is bound to HrtR, HrtR cannot bind to hrtO and therefore, downstream transcription proceeds. Conversely, when heme is not bound to HrtR, HrtR can bind to hrtO and inhibit downstream transcription. FIG. 1A illustrates the binding of HrtR to hrtO in the absence of heme and FIG. 1B illustrates the binding of heme to an HrtR protein, thereby releasing HrtR from hrtO. In order for heme to influence the binding of HrtR to hrtO, HrtR must be sufficiently present to favor said binding.

The particular nucleotide sequence of hrtO and the amino acid sequence of HrtR vary from bacterial strain to bacterial strain and therefore the exact sequence of each may be chosen based on the intended bacteria strain into which the heme-responsive repressor system will be introduced. In any embodiment, a heme-responsive repressor system should encode for expression of an HrtR protein that reversibly binds to the hrtO repressor binding site in the heme-responsive promoter depending on the co-binding of heme.

Therefore, provided herein is a heme-responsive repressor systems comprising the heme-responsive promoter and a sequence that encodes expression of an HrtR protein that binds to the hrtO in the heme-responsive promoter. In particular, a heme-responsive repressor system may comprise a) a first nucleic acid comprising a heme-responsive promoter comprising a constitutive promoter modified with one or more hrtO repressor binding sites and b) a second nucleic acid that encodes for the expression of an HrtR protein that binds the one or more hrtO repressor binding sites in the first nucleic acid. The second nucleic acid may comprise any constitutive promoter that is operably linked to a gene encoding for the HrtR protein. According to the present disclosure, the constitutive promoter driving the expression of the HrtR protein is effective to provide a sufficient amount of HrtR to favor binding to the hrtO transcriptional repressor binding site when HrtR is not bound to heme.

For example, the second nucleic acid may induce expression of an HrtR repressor protein having an amino sequence represented by any one of SEQ ID NOs:1-100, as shown below in Table 1. SEQ ID NOs:1-56 are from Lactococcus species of bacteria and will bind with high affinity to a Lactococcus hrtO repressor binding site, however the remaining SEQ ID NOs:57-100 have sufficient homology to allow binding as well. Additionally, the HrtR proteins represented by SEQ ID NOs:57-100 may be utilized in a heme-responsive repressor binding system for use in their endogenous bacterial strain. For example, SEQ ID NO:60 represents an HrtR repressor binding protein from Bacillus cereus. A heme-responsive repressor binding system for use in Bacillus cereus may comprise a first nucleic acid comprising a heme-responsive promoter comprising a constitutive promoter from Bacillus cereus modified with one or more hrtO repressor binding sites from Bacillus cereus; and a second nucleic acid encoding for an HrtR protein represented by SEQ ID NO:60.

| ID | Accession | | |
|---|---|---|---|
| No: | No. | Sequence | |
| 1 | WP_ | 1 | MPKSTYFSLS DEKRKRVYDA CLLEFQTHSF HEAKIMHIVK ALDIPRGSFY QYFEDLKDSY |
| | 011834636.1 | 61 | YYILSQETVE IHDLFFNLLK EYPLEVALNK YKYLLLENLV NSPQYNLYKY RFLDWTYELE |
| | | 121 | RDWKPKGEVT VPARELDNPI SQVLKSVIHN LVYRMFSENW DEQKFIETYD KEIKLLTEGL |
| | | 181 | LNYVTESKK |
| 2 | WP_ | 1 | MPKSTYFSLS DEKRKRVYDA CLLEFQTHSF HEAKIMHIVK ALDIPRGSFY QYFEDLKDSY |
| | 063281424.1 | 61 | YYILSQETVE IHDLFFNLLK EYPLEVSLNK YKYLLLENLV NSPQYNLYKY RFLDWTYELE |
| | | 121 | RDWKPKGEVT VPARELDNPI SQVLKSVIHN LVYRMFSENW NEQKFIETYD KEIKLLTEGL |
| | | 181 | LNYVTESKK |
| 3 | WP_ | 1 | MPKSTYFSLS DEKRKRVYDA CLLEFQTHSF HEAKIMHIVK ALDIPRGSFY QYFEDLKDSY |
| | 021046844.1 | 61 | YYILSQETVE IHDLFFNLLK EYPLEVSLNK YKYLLLENLV NSPQYNLYKY RFLDWTYELE |
| | | 121 | RDWKPKGEVT VPTRELDNPI SQVLKSVIHN LVYRMFSENW DEQKFIETYD KEIKLLTEGL |
| | | 181 | LNYVTESKK |
| 4 | WP_ | 1 | MPKSTYFSLS DEKRKRVYDA CLLEFQTHSF HEAKIMHIVK ALDIPRGSFY QYFEDLKDSY |
| | 014573008.1 | 61 | YYILSQETVE IHDLFFNLLK EYPLEVALDK YKYLLLENLV NSPQYNLYKY RFLDWTYELE |
| | | 121 | RDWKPKGEVT VPARELDNPI SQVLKSVIHN LVYRMFSENW NEQKFIETYD KEIKLLTEGL |
| | | 181 | LNYVTESKK |
| 5 | PCS14840.1 | 1 | MIGEKMPKST YFSLSDEKRK RVYDACLLEF QTHSFHEAKI MHIVKALDIP RGSFYQYFED |
| | | 61 | LKDSYYYILS QETVEIHDLF FNLLKEYPLE VALDKYKYLL LENLVNSPQY NLYKYRFLDW |
| | | 121 | TYELERDWKP KGEVTVPARE LDNPISQVLK SVIHNLVYRM FSENWNEQKF IETYDKEIKL |
| | | 181 | LTEGLLNYVT ESKK |
| 6 | WP_ | 1 | MPKSTYFSLS DEKRKRVYDA CLLEFQTHSF HEAKIMHIVK ALDIPRGSFY QYFEDLKDSY |
| | 011675628.1 | 61 | YYILSQETVE IHDLFFNLLK EYPLEVALDK YKYLLLENLV NSPQYNLYKY RFLDWTYELE |
| | | 121 | RDWKPKGEVT VPARELDNPI SQVLKSVIHN LVYRMFSENW NEQKFIETYD KEIKLLTEGL |
| | | 181 | LNYVTENKK |
| 7 | PCS18458.1 | 1 | MIGEKMPKST YFSLSDEKRK RVYDACLLEF QTHSFHEAKI MHIVKALDIP RGSFYQYFED |
| | | 61 | LKDSYYYILS QETVEIHDLF FNLLKEYPLE VSLDKYKYLL LENLVNSPHY NLYKYRFLDW |
| | | 121 | TYELERDWKP KGEVTVPARE LDNPISQVLK SVIHNLVYRM FSENWNEQKF IETYDKEIKL |
| | | 181 | LTEGLLNYVT ESKK |
| 8 | WP_ | 1 | MPKSTYFSLS DEKRKRVYDA CLLEFQTHSF HEAKIMHIVK ALDIPRGSFY QYFEDLKDSY |
| | 043736022.1 | 61 | YYILSQETVE IHDLFFNLLK EYPLEVSLDK YKYLLLENLV NSPHYNLYKY RFLDWTYELE |
| | | 121 | RDWKPKGEVT VPARELDNPI SQVLKSVIHN LVYRMFSENW NEQKFIETYD KEIKLLTEGL |
| | | 181 | LNYVTESKK |
| 9 | WP_ | 1 | MPKSTYFSLS DEKRNRVYDA CLNEFQTHSF HEAKIMHIVK ALDIPRGSFY QYFEDLKDAY |
| | 023188929.1 | 61 | FYVLSQETLE IHDLFFNLLK DYSIEEALDK YKYLLLENLI DSPQYQLYKY RFLDWTYELE |
| | | 121 | RDWKPKSSAT VPASENNNPV SQVLKSVVHN LVYRLFSENW TEKTFIENYD KEIKLVTEGL |
| | | 181 | LNYITDRKN |
| 10 | WP_ | 1 | MPKSTYFSLS DEKRNRVYDA CLNEFQTHSF HEAKIMHIVK ALDIPRGSFY QYFEDLKDAY |
| | 085624040.1 | 61 | FYVLSQETLE IHDLFFNLLK DNSIEEALDK YKYLLLENLI DSPQYKLYKY RFLDWTYELE |
| | | 121 | RDWKPKSSAT VPASENDNPI SQVLKSVVHN LVYRLFSENW TEKTFIENYD KEIKLVTEGL |
| | | 181 | LNYITDRKN |
| 11 | WP_ | 1 | MPKSTYFSLS DEKRNRVYDA CLNEFQTHSF HEAKIMHIVK ALDIPRGSFY QYFEDLKDAY |
| | 047205779.1 | 61 | FYVLSQETLE IHDLFFNLLK DNSIEEALDK YKYLLLENLI DSPQYQLYKY RFLDWTYELE |
| | | 121 | RDWKPKSSAT VPASENDNPV SQVLKSVVHN LVYRLFSENW TEKTFIENYD KEIKLVTEGL |
| | | 181 | LNYITDRKN |
| 12 | WP_ | 1 | MPKSTYFSLS DEKRNRVYDA CLNEFQTHSF HEAKIMHIVK ALDIPRGSFY QYFEDLKDAY |
| | 012897348.1 | 61 | FYVLSQETLE IHDLFFNLLK DNSIEEALDK YKYLLLENLI DSPQYQLYKY RFLDWTYELE |
| | | 121 | RDWKPQSSAT VPASENDNPI SQVLKSVVHN LVYRLFSENW TEKTFIENYD KEIKLVTEGL |
| | | 181 | LNYITDRKN |
| 13 | WP_ | 1 | MPKSTYFSLS DEKRNRVYDA CLNEFQTHSF HEAKIMHIVK ALDIPRGSFY QYFEDLKDAY |
| | 129881475.1 | 61 | FYVLSQETLE IHDLFFNLLK DNSIEEALDK YKYLLLENLI DSPQYQLYKY RFLDWTYELE |
| | | 121 | RDWKPQSSAT VPASENDNPI SQVLKSVVHN LVYRLFSENW TEKNFIENYD KEIKLVTEGL |
| | | 181 | LNYITDRKN |
| 14 | WP_ | 1 | MPKSTYFSLS DEKRNRVYDA CLNEFQTHSF HEAKIMHIVK ALDIPRGSFY QYFEDLKDAY |
| | 017864218.1 | 61 | FYVLSQETLE IHDLFFNLLK DNSIEEALDK YKYLLLENLI DSPQYKLYKY RFLDWTYELE |
| | | 121 | RDWKPQSSAT VPASENDNPI SQVLKSVVHN LVYRLFSENW TEKTFIENYD KEIKLVTEGL |
| | | 181 | LNYITDRKN |
| 15 | WP_ | 1 | MPKSTYFSLS DEKRNRVYDA CLNEFQTHSF HEAKIMHIVK ALDIPRGSFY QYFEDLKDAY |
| | 098383614.1 | 61 | FYVLSQETLE IHDLFFNLLK DNSVEESLDK YKYLLLENLI DSPQYQLYKY RFLDWTYELE |
| | | 121 | RDWKPKSSAT VPASENDNPI SQVLKSVVHN LVYRLFSENW TEKTFIENYD KEIKLVTEGL |
| | | 181 | LNYITDRKN |
| 16 | KAA8700799.1 | 1 | MPKSTYFSLS DEKRNRVYDA CLNEFQTHSF HEAKIMHIVK ALDIPRGSFY QYFEDLKDAY |
| | | 61 | FYVLSQETLE IHDLFFNLLK DNSIEEALDK YKYLLLENLI DSPQYQLYKY RFLDWTYDLE |
| | | 121 | RDWKPKSSAT VPASENDNPV SQVLKSVVHN LVYRLFSENW TEKTFIENYD KEIKLVTEGL |
| | | 181 | LNYITDRKN |
| 17 | WP_ | 1 | MPKSTYFSLS DEKRNRVYDA CLNEFQTHSF HEAKIMHIVK ALDIPRGSFY QYFEDLKDAY |
| | 033899642.1 | 61 | FYVLSQETLE IHDLFFNLLK DNSIEEALDK YKYLLLENLI DSPQYQLYRY RFLDWTYELE |
| | | 121 | RDWKPQSSAT VPASENDNPI SQVLKSVVHN LVYRLFSENW TEKTFIENYD KEIKLVTEGL |
| | | 181 | LNYITDRKN |
| 18 | WP_ | 1 | MPKSTYFSLS DEKRNRVYDA CLNEFQTHSF HEAKIMHIVK ALDIPRGSFY QYFEDLKDAY |
| | 039114777.1 | 61 | FYVLSQETLE IHDLFFNLLK DNSIEEALDK YKYLLLENLI DSPQYQLYKY RFLDWTYELE |
| | | 121 | RDWKPKSSAT VPASENNNPV SQVLKSVVHN LVYRLFSENW TEKTFIENYD KEIKLVTEGL |
| | | 181 | LNYITDRKN |
| 19 | WP_ | 1 | MPKSTYFSLS EEKRNRVYDA CLNEFQTHSF HEAKIMHIVK ALDIPRGSFY QYFEDLKDAY |
| | 104054514.1 | 61 | FYVLSQETLE IHDLFFNLLK DNSIEEALDK YKYLLLENLI DSPQYQLYKY RFLDWTYELE |
| | | 121 | RDWKPKSSAT VPASENDNPV SQVLKSVVHN LVYRLFSENW TEKTFIENYD KEIKLVTEGL |
| | | 181 | LNYITDRKN |
| 20 | WP_ | 1 | MPKSTYFSLS DEKRNRVYDA CLNEFQTHSF HEAKIMHIVK ALDIPRGSFY QYFEDLKDAY |
| | 195935592.1 | 61 | FYVLSQETLE IHELFFNLLK DNSIEEALDK YKYLLLENLI DSPQYQLYKY RFLDWTYELE |
| | | 121 | RDWKPKSSAT VPASENDNPV SQVLKSVVHN LVYRLFSENW TEKTFIENYD KEIKLVTEGL |
| | | 181 | LNYITDRKN |
| 21 | WP_ | 1 | MPKSTYFSLS DEKRNRVYDA CLNEFQTHSF HEAKIMHIVK ALDIPRGSFY QYFEDLKDAY |
| | 010905492.1 | 61 | FYVLSQETLE IHDLFFNLLK DNSIEESLDK YKYLLLENLI DSPQYKLYKY RFLDWTYELE |
| | | 121 | RDWKPQSSAT VPASENDNPI SQVLKSVVHN LVYRLFSENW TEKTFIENYD KEIKLVTEGL |
| | | 181 | LNYITDRKN |
| 22 | WP_ | 1 | MPKSTYFSLS DEKRNRVYDA CLNEFQTHSF HESKIMHIVK ALDIPRGSFY QYFEDLKDAY |
| | 098396281.1 | 61 | FYVLSQETLE IHDLFFNLLK DNSIEEALDK YKYLLLENLI DSPQYKLYKY RFLDWTYELE |
| | | 121 | RDWKPQSSAT VPASENDNPI SQVLKSVVHN LVYRLFSENW TEKTFIENYD KEIKLVTEGL |
| | | 181 | LNYITDRKN |
| 23 | WP_ | 1 | MPKSTYFSLS DEKRNRVYDA CLNEFQTHSF HEAKIMHIVK ALDIPRGSFY QYFEDLKDAY |
| | 038599617.1 | 61 | FYVLSQETLE IHDLFFNLLK DNSIEEALDK YKSLLLENLI DSPQYQLYKY RFLDWTYELE |
| | | 121 | RDWKPKSSAT VPASENDNPV SQVLKSVVHN LVYRLFSENW TEKTFIENYD KEIKLVTEGL |
| | | 181 | LNYITDRKN |
| 24 | WP_ | 1 | MPKSTYFSLS DEKRNRVYDA CLNEFQTHSF HEATIMHIVK ALDIPRGSFY QYFEDLKDAY |
| | 058224287.1 | 61 | FYVLSQETLE IHDLFFNLLK DNSIEEALDK YKYLLLENLI DSPQYKLYKY RFLDWTYELE |
| | | 121 | RDWKPQSSAT VPASENDNPI SQVLKSVVHN LVYRLFSENW TEKTFIENYD KEIKLVTEGL |
| | | 181 | LNYITDRKN |
| 25 | WP_ | 1 | MPKSTYFSLS DEKRNRVYDA CLNEFQTHSF HETKIMHIVK ALDIPRGSFY QYFEDLKDAY |
| | 058206834.1 | 61 | FYVLSQETLE IHDLFFNLLK DNSIEEALDK YKYLLLENLI DSPQYKLYKY RFLDWTYELE |
| | | 121 | RDWKPQSSAT VPASENDNPI SQVLKSVVHN LVYRLFSENW TEKTFIENYD KEIKLVTEGL |
| | | 181 | LNYITDRKN |
| 26 | WP_ | 1 | MPKSTYFSLS DEKRNRVYDA CLNEFQTHSF HEAKIMHIVK ALDIPRGSFY QYFEDLKDAY |
| | 015425958.1 | 61 | FYVLSQETLE IHDLFFNLLK DNSIEEALDK YKYLLLENLI DSPQYQLYKY RFLDWTCELE |
| | | 121 | RDWKPKSSAT VPASENDNPV SQVLKSVVHN LVYRLFSENW TEKTFIENYD KEIKLVTEGL |
| | | 181 | LNYITDRKN |
| 27 | WP_ | 1 | MPKSTYFSLS DEKRNRVYDA CLNEFQTHSF HEAKIMHIVK ALDIPRGSFY QYFEDLKDAY |
| | 153241919.1 | 61 | FYVLSQETLE IHELFFNLLK DNSIEEALDK YKYLLLENLI DSPQYQLYKY RFLDWTYELE |
| | | 121 | RDWKPQSSAT VPASENDNPV SQVLKSVVHN LVYRLFSENW TEKTFIENYD KEIKLVTEGL |
| | | 181 | LNYITDRKN |
| 28 | WP_ | 1 | MPKSTYFSLS DEKRNRVYDA CLNEFQTHSF HEAKIMHIVK ALDIPRGSFY QYFEDLKDAY |
| | 081213416.1 | 61 | FYVLSQETLE IHDLFFNLLK DNSIEEALYK YKYLLLENLI DSPQYKLYKY RFLDWTYELE |
| | | 121 | RDWKPQSSAT VPASENDNPI SQVLKSVVHN LVYRLFSENW TEKTFIENYD KEIKLVTEGL |
| | | 181 | LNYITDRKN |
| 29 | WP_ | 1 | MPKSTYFSLS DEKRNRVYDA CLNEFQTHSF HEAKIMHIVK ALDIPRGSFY QYFEDLKDAY |
| | 168784794.1 | 61 | FYVLSQETLE IHDLFFNLLK DNSIEEALDK YKYLLLENLI DSPQYQLYKY RFLDWTYELE |
| | | 121 | RDWKSQSSAT VPASENDNPI SQVLKSVVHN LVYRLFSENW TEKNFIENYD KEIKLVTEGL |
| | | 181 | LNYITDRKN |
| 30 | WP_ | 1 | MPKSTYFSLS DEKRNRVYDA CLNEFQTHSF HEAKIMHIVK ALDIPRGSFY QYFEDLKDAY |
| | 075524485.1 | 61 | FYVLSQETLE IHDLFFNLLK DNSIEEALDK YKYLLLENLI DSPQYQLYKY RFLDWTYELE |
| | | 121 | RDWKPKSSAT VPASENDNPV SQVLKSVVHN LVYRLFSENW TKKTFIENYD KEIKLVTEGL |
| | | 181 | LNYITDR |
| 31 | WP_ | 1 | MPKSTYFSLS DEKRNRVYDA CLNEFQTHSF HEAKIMHIVK ALDIPRGSFY QYFEDLKDAY |
| | 124137335.1 | 61 | FYVLSQETLE IHDLFFNLLK DNSIEEVLDK YKYLLLENLI DSPQYQLYKY RFLDWTYELE |
| | | 121 | RDWKPKSSAT VPASENNNPV SQVLKSVVHN LVYRLFSENWT EKTFIENYD KEIKLVTEGL |
| | | 181 | LNYITDRKN |
| 32 | WP_ | 1 | MPKSTYFSLS DEKRNRVYDA CLNEFQTHSF HEAKIMHIVK ALDIPRGSFY QYFEDLKDAY |
| | 139918039.1 | 61 | FYVLSQETLE IHDLFFNLLK DNSIEEALDK YKYLLLENLI DSPQYQLYKC RFLDWTYELE |
| | | 121 | RDWKPQSSAT VPASENDNPV SQVLKSVVHN LVYRLFSENW TEKTFIENYD KEIKLVTEGL |
| | | 181 | LNYITDRKN |
| 33 | WP_ | 1 | MPKSTYFSLS DEKRNRVYDA CLNEFQTHSF HEAKIMHIVK ALDIPRGSFY QYFEDLKDAY |
| | 137016540.1 | 61 | FYVLSQETLE IHDLFFNLLK YNSIEESLDK YKYLLLENLI DSPQYKLYKY RFLDWTYELE |
| | | 121 | RDWKPQSSAT VPASENDNPI SQVLKSVVHN LVYRLFSENW TEKTFIENYD KEIKLVTEGL |
| | | 181 | LNYITDRKN |
| 34 | WP_ | 1 | MPKSTYFSLS DEKRNRVYDA CLNEFQTHSF HEAKIMHIVK ALDIPRGSFY QYFEDLKDAY |
| | 029344949.1 | 61 | FYVLSQETLE IHELFFNLLI DNSIEEALDK YKYLLLENLI DSPQYQLYKY RFLDWTYELE |
| | | 121 | RDWKPKSSAT VPASENDNPV SQVLKSVVHN LVYRLFSENW TEKTFIENYD KEIKLVTEGL |
| | | 181 | LNYITDRKN |
| 35 | WP_ | 1 | MPKSTYFSLS DEKCNRVYDA CLNEFQTHSF HEAKIMHIVK ALDIPRGSFY QYFEDLKDAY |
| | 058225960.1 | 61 | FYVLSQETLE IHDLFFNLLK DNSIEESLDK YKYLLLENLI DSPQYKLYKY RFLDWTYELE |
| | | 121 | RDWKPQSSAT VPASENDNPI SQVLKSVVHN LVYRLFSENW TEKTFIENYD KEIKLVTEGL |
| | | 181 | LNYITDRKN |
| 36 | WP_ | 1 | MPKSTYFSLS DEKRNRVYDA CLNEFQTHSF HEAKIMHIVK ALDIPRGSFY QYFEDLKDAY |
| | 003129571.1 | 61 | FYVLSKETLE IHDLFFNLLK YNSIEESLDK YKYLLLENLI DSPQYKLYKY RFLDWTYELE |
| | | 121 | RDWKPQSSAT VPASENDNPI SQVLKSVVHN LVYRLFSENW TEKTFIENYD KEIKLVTEGL |
| | | 181 | LNYITDRKN |
| 37 | WP_ | 1 | MPKSTYFSLS DEKRNRVYDA CLNEFQTHSF HEAKIMHIVK ALDIPRGSFY QYFEDLKDAY |
| | 200407693.1 | 61 | FYVLSQETLE IHDLFFNLLK DNSIEEALDK YKYLLLENLI DSPQYQLYKY RFLDWTYELE |
| | | 121 | RDWKPKSSAT VPASENDNPD SQVLKSVVHN LVYRLFSENW TKKTFIENYD KEIKLVTAGL |
| | | 181 | LNYITDR |
| 38 | KSU30961.1 | 1 | MHIVKALDIP RGSFYQYFED LKDAYFYVLS QETLEIHDLF FNLLKDNSIE ESLDKYKYLL |
| | | 61 | LENLIDSPQY KLYKYRFLDW TYELERDWKP QSSATVPASE NDNPISQVLK SVVHNLVYRL |
| | | 121 | FSENWTEKTF IENYDKEIKL VTEGLLNYIT DRKN |
| 39 | WP_ | 1 | MPKETYFSLP GEKRNRVYQA CKLEFETHSY QDAKVMHVVK ALNISRGSFY QYFEDLKDCY |
| | 096818096.1 | 61 | FFVLSNETIE LHQLFIDLVK VYSIDEALEK YKFLLLEQLI RGENYEIYRH RFLDWNYDLD |
| | | 121 | HAWKQKATGT FHSSKSNNPI TQVLKSVVHD LVYRLFSENW DEETFIEHYE KEIEVVCIGL |
| | | 181 | EAYYKNMKSH H |
| 40 | WP_ | 1 | MPKETFFSLP DEKRMRVYQA CKQEFEQHPF HEAKVMHIVK ALGIPRGSFY QYFDDLMECY |
| | 153496858.1 | 61 | FYILSKETKE LHGAFIDLIK VYPFEEAIDR YKYLLLEQLI HSDQYALYRY RFLSWDYRLD |
| | | 121 | QAWAVRFGGT YHSEQKENPV IQVFKAVVHD LVYRLYAKAW DDQAFIAHYD RELSVVLNGV |
| | | 181 | KHEMMGHPHE KRIKE |
| 41 | WP_ | 1 | MPKETYFSLP DEKRMRVYQA CKQEFEKYSF HDAKIMHIVK ALDIPRGSFY QYFEDLTDCY |
| | 147779743.1 | 61 | FYVLSKETTE LHEIFMELIK HYSLDEALER YKYLLVDELL HGERASLYRF RFLDWNYELD |
| | | 121 | QAWSERMVTS FEKERKNNAT IQVFKAVVHD LIYQLYSKNW DATTFIAHYD SEISVLINGI |
| | | 181 | NTLTKVEKQE |
| 42 | WP_ | 1 | MPKATYLKLP EEKKQRIYAA CKTEFADKPF HQATVRNIVQ SLEIPRGSFY QYFEDLTDCY |
| | 195917338.1 | 61 | IYVLSQETSE LHQLFLDVIK KYPLEKALDE YKHLLLKELV HNENYAIYKY RFLDWNHELT |
| | | 121 | LAWKKYNPQT FVSKEPVSPM ISVFKAVVHD LVYRLFAEEW SDQEFVAHYD SEVAVIMNGL |
| | | 181 | SGSQNHQ |
| 43 | WP_ | 1 | MPKATYLKLP EEKKQRIYAA CKTEFADKPF HQATVRNIVQ SLEIPRGSFY QYFEDLTDCY |
| | 014025275.1 | 61 | IYVLSQETSE LHQLFLDAIK KYPLEKALDE YKHLLLQELV HNENYAIYKY RFLDWNHELT |
| | | 121 | LAWKKYNPQT FVSKEPVSPM ISVFKAVVHD LVYRLFAEEW SDQEFVAHYD SEVAVIMNGL |
| | | 181 | SGSQNHQ |
| 44 | HAP15461.1 | 1 | MPKATYLKLP EEKKQRIYAA CKTEFADKPF HQATVRNIVQ SLDIPRGSFY QYFEDLTDCY |
| | | 61 | MYVLSQETRE LHQLFLDAIQ KYPLEQALDE YKHLLLKELV HNENYAIYKY RFLDWNHELT |
| | | 121 | LAWKKYNPQT FVSKEPVSPM ISVFKAVVHD LVYRLFAEEW SDQEFVAHYD SEVAVIMNGL |
| | | 181 | SGSQNHQ |
| 45 | WP_ | 1 | MPKATYLKLP EEKKQRIYAA CKTEFADKPF HQATVRNIVQ SLEIPRGSFY QYFEDLTDCY |
| | 064305335.1 | 61 | IYVLSQETSE LHQLFLDAIK KYPLEKALDE YKHLLLKELV HNENYAIYKY RFLDWNHELT |
| | | 121 | LAWKKYNPQT FVSKEPVSPM ISVFKAVVHD LVYRLFAEEW GDQEFVAHYD SEVAVIMKGL |
| | | 181 | SGSQNHQ |
| 46 | WP_ | 1 | MPKATYLKLP EEKKQRIYAA CKTEFADKPF HQATVRNIVQ SLEIPRGSFY QYFEDLTDCY |
| | 003134238.1 | 61 | IYVLSQETSE LHQLFLDAIK KYPLEKALDE YKHLLLKELV HNENYAIYKY RFLDWNHELT |
| | | 121 | LAWKKYNPQT FVSKEPVSPM ISVFKAVVHD LVYRLFAEEW SDQEFVAHYD SEVAVIMNGL |
| | | 181 | SGSQNHQ |
| 47 | WP_ | 1 | MPKATYLKLP EEKKQRIYAA CKTEFADKPF HQATVRNIVQ SLEIPRGSFY QYFEDLTDCY |
| | 074751496.1 | 61 | IYVLSQETSE LHQLFLEAIK KYPLEKALDE YKHLLLQELV HNENYAIYKY RFLDWNHELT |
| | | 121 | LAWKKYNPQT FVSKEPVSPM ISVFKAVVHD LVYRLFAEEW SDQEFVAHYD SEVAVIMNGL |
| | | 181 | SGSQNHQ |
| 48 | WP_ | 1 | MPKATYLKLP EEKKQRIYAA CKTEFADKPF HQATVRNIVQ SLEIPRGSFY QYFEDLTDCY |
| | 141921128.1 | 61 | IYVLSQETSE LHQLFLEAIK KYPLEKALDE YKHLLLKELV HNENYAIYKY RFLDWNHELT |
| | | 121 | LAWKKYNPQT FVSKEPVSPM ISVFKAVVHD LVYRLFAEEW SDQEFVAHYD SEVAVIMNGL |
| | | 181 | SGSQNHQ |
| 49 | WP_ | 1 | MPKATYLKLP DEKKQRIYAA CKTEFADKPF HQATVRNIVQ SLDIPRGSFY QYFEDLTECY |
| | 096369162.1 | 61 | MYVLSKETSE LHQLFLDAIQ KYPLEQALDE YKHLLLKELV HNENYAIYKY RFLDWNHELT |
| | | 121 | LAWKKYNPQT FVSKEPVSPM ISVFKAVVHD LVYRLFAEEW SDQEFVAHYD SEVGVIMKGL |
| | | 181 | SGSQNHQ |
| 50 | WP_ | 1 | MPKATYLKLP DEKKQRIYAA CKTEFADKPF HQATVRNIVQ SLDIPRGSFY QYFEDLTECY |
| | 165714825.1 | 61 | MYVLSKETSE LHQLFLDAIQ KYPLEQALDE YKHLLLKELV HNENYAFYKY RFLDWNHELS |
| | | 121 | LAWKKYNPQT FVSKEPVSPM ISVFKAVVHD LVYRLFAEEW SDQEFVAHYD SEVGVIMKGL |
| | | 181 | SGSQNHQ |
| 51 | WP_ | 1 | MPKATYLKLP EEKKQRIYAA CKTEFADKPF HQATVRNIVQ SLEIPRGSFY QYFEDLTDCY |
| | 086583047.1 | 61 | IYVLSQETSE LHQLFLDAIT KYPLEKALDE YKHLLLKELV HNENYAIYKY RFLDWNHELT |
| | | 121 | LAWKKYNPQT FVSKEPVSPM ISVFKAVVHD LVYRLFAEEW SDQEFVAHYD SEVAVIMNGL |
| | | 181 | SGSQNHQ |
| 52 | WP_ | 1 | MPKATYLKLP EEKKQRIYAA CKTEFADKPL HQATVRNIVQ SLEIPRGSFY QYFEDLTDCY |
| | 117517698.1 | 61 | IYVLSQETSE LHQLFLDAIK KYPLEKALDE YKHLLLKELV HNENYAIYKY RFLDWNHELT |
| | | 121 | LAWKKYNPQT FVSKEPVSPM ISVFKAVVHD LVYRLFAEEW SDQEFVAHYD SEVAVIMNGL |
| | | 181 | SGSQNHQ |
| 53 | WP_ | 1 | MPKATYLKLP EEKKQRIYAA CKTEFADKPF HQATVRNIVQ SLDIPRGSFY QYFEDLTECY |
| | 017370213.1 | 61 | MYVLSKETSE LHQLFLDAIQ KYPLEQALDE YKHLLLKELV HNENYAIYKY RFLDWNHELT |
| | | 121 | LAWKKYNPQT FVSKEPVSPM ISVFKAVVHD LVYRLFAEEW SDQEFVAHYD SEVGVIMKGL |
| | | 181 | SGSQNHQ |
| 54 | WP_ | 1 | MPKATYLKLP EEKKQRIYAA CKTEFADKPF HQATVRNIVQ SLDIPRGSFY QYFEDLTECY |
| | 165713737.1 | 61 | MYVLSKETSE LHQLFLDAIQ KYPLEQALDE YKHLLLKELV HNENYAIYKY RFLDWNHELT |
| | | 121 | LAWKKYNPQT FVSKEPVSPM ISVFKAVVHD LVYRLFAEEW SDQEFVAHYD SEVGVIMNGL |
| | | 181 | SGSQNHQ |
| 55 | WP_ | 1 | MPKATYLKLP ESKKQRIYAV CKKEFSDKPF HQATVRDIVQ ALEIPRGSFY QYFEDLTDCY |
| | 003136031.1 | 61 | MYVLSQETKE LHNLFIDLIQ KLPLMEALEE YKHLLLKELV HNENYSLYKY RFLDWNYELS |
| | | 121 | LAWKKYNHQT FVTKEPVSPM ISVFKSVVHD LVYRLFAEEW DDNEFITHYD NEIAIIMNGV |
| | | 181 | SMSHIHH |
| 56 | WP_ | 1 | MPKATYLKLP ESKKQRIYAV CKKEFSDKPF HQATVRDIVQ ALEIPRGSFY QYFEDLTDCY |
| | 081168467.1 | 61 | MYVLSQETKE LHNLFIDLIQ KLPLMEALEE YKHLLLKELV HNENYSLYKY RFLDWNYELS |
| | | 121 | LAWKKYNHQT FVTKEPVSPM ISVFKSVVHD LVYRLFAEEW DDNEFITHYD NEIAIIMNGV |
| | | 181 | SMSHIHH |
| 57 | WP_ | 1 | MPKETYYYLP DEKKTRVYLA CRSEFETHSY HEAKIMHIVK ALNIPRGSFY QYFDDLKDCY |
| | 183540127.1 | 61 | FYVLSKETIE IHDLFFELIK KHSLEESLEK YKYILLENLL HSSSYNLYRY RFLDWNYELE |
| | | 121 | KDWKPKGTKT VSSVELNNPL IQVLKAVIHD IVHRLFSEKW SDEEFITVYN REIGLLKQGL |
| | | 181 | TAYLSHG |
| 58 | MBD4919663.1 | 1 | KYLVLENLVN SPQYNLYKYR FLDWTYELER DWKPKGEVTV PARELDNPIS QVLKSVIHNL |
| | | 61 | VYRMFSENWD EQKFIETYDK EIKLLTEGLL NYVSESKK |
| 59 | RRC92211.1 | 1 | MPKETYFHLS KEKQQRIYEV CKQEFETHSF YEAKVSHIVK ALGISRGSFY LYFEDLQDCY |
| | | 61 | FYILQKETCE LHDLFMQLMD HEDVVNALRK YQDVLLAHLL DPSVKRLYET RFLYWGHDLN |
| | | 121 | QAWKAYQVTS STFSPMPSDF IHYLKAVIHD LVFRLYSEGW DAETFKQHYQ TVMHYVLYGI |
| | | 181 | SHRKEHAHEH HQ |
| 60 | WP_ | 1 | MPKSTYFSLS DEKRNRVYDA CLNEFQTHSF HEAKIMHIVK ALDIPRGSFY QYFEDLKDAY |
| | 141520352.1 | 61 | FYVLSQETLE IHDLFFNLLK DNSIEEAL |
| 61 | PID81170.1 | 1 | MSLSGGVMPK KTFNNLSYER QQHVLAICRR EFETHCFDAA SVSAIVEQLG IARGTFYKYF |
| | | 61 | EDLEDCYFYL LARETKEVHD IFFFLLKQNE FDIVTSLAQY GKRIAREIHD PQKYALYRNR |
| | | 121 | YLGWTPLLQE KWQIYLEKLG KKAMPFADVV VEGYRAELLS GESIHLIKAV VHDLIARNFS |
| | | 181 | ENWTKAHFIK NYQRHIQLLT EGLKSNLLRD |
| 62 | WP_ | 1 | MPKATFYNLS EEKQARILEV LKHTFQTKTI FEANVKEIVE ASEIARGSFY QYFENLEEAY |
| | 126465686.1 | 61 | FTILDRETVD IHDLFMALLT EQEGDVFAAL EEYGDRLAEI LFVEGNYGIY KNRFLYWTPG |
| | | 121 | LEEGWQAYRK SAAAGASDRD RYLYREEMQF IKAVVHNLIQ RNFLEKWDAQ EFVRHYKQYA |
| | | 181 | AWLKGGVHID GTR |
| 63 | ERJ82248.1 | 1 | MPKTTFHHLS EEKRQAVVAV LIHEFESYPL PDATVKHIVD ALGIARGSFY QYFDSLTESY |
| | | 61 | FYVLEREVIE THSLFLKLFH QCHRDTMQAL EIYGEVLAKE IFQGKHYALY KNRYLYWNAS |
| | | 121 | LERDWRNYLA ENQKDSSPME SMNENEPIRF VKAVIHALMQ RLFMENWDRE TFLKRYHQHI |
| | | 181 | QWIKGGLTNVSPD |
| 64 | WP_ | 1 | MPKNTFYNLP EEKKSRVVGV LKETFSAKSI YQANVKEIVE KLDIARGSFY KYFEDIEDAY |
| | 075574183.1 | 61 | FTILDMETAD IHKVFLDFFK ENDHDIHKAL EAYGKALTEI LFEPDTYGLY KNRYLNWTPD |
| | | 121 | LEGKWRKYLK EFRPGEDDFS ASDFDKDRLE FMKAAVHNLI QRTYLEAWDR EEFLNHYNKY |
| | | 181 | IAWLKGGI |
| 65 | WP_ | 1 | MPKNTFYNLP EEKKSRVVGI LKETFSAKSI YQANVKEIVE KLDIARGSFY KYFEDIEDAY |
| | 099951263.1 | 61 | FTILDMETAD IHKLFLSLLE KNNFDIHKAL EAYGNALAEI LFEPDTYGLY KNRYLNWTPD |
| | | 121 | LEGKWRKYLK EFRPGEDDFS TSDFDRDRLE FMKAAVHNLI QRTYLEAWDR EEFLNHYNKY |
| | | 181 | IAWLKGGI |
| 66 | WP_ | 1 | MPKATFFNLS NEKRKKIVKV LTEEFEKKNI QEATVKEIVQ KLGIARGSFY QYFETLEESY |
| | 046328791.1 | 61 | FYILELKTAD LHKSFMNLLV KYGDIYDVLE EYGEKIVEII FDKSCYNLYK NRYLLWNEKI |
| | | 121 | NRQWEEYKQK NMINLIEVRN TDELLYISAL MHSLISRSYI EKWDKDKFLE KYKKYVRWIR |
| | | 181 | EGVRDE |
| 67 | MBE2989314.1 | 1 | MPKVTFFNLS KEKRKKIVEV LTEEFEKKNI QEATVKEIVQ KLGIARGSFY QYFETLEESY |
| | | 61 | FYILELKTAD LHKSFMNLLV KYGDIYDVLG EYGEKIVEII FDKSCYNLYK NRYLLWNEKI |
| | | 121 | NRQWEEYKQK NMTSLIEVRN TDELLYISAL MHSLISRSYI EKWDKDKFLE KYKKYVKWIK |
| | | 181 | EGVRDE |
| 68 | CAC9936055.1 | 1 | MPRSTFFSLK AEKRERIEKA LVSEFENRTL GDAKVKHVVE RLGISRGSFY QYFHNMQDAY |
| | | 61 | FYILDLKLRF IHEGFFTLYR AEPQNIESVL EKYGVFLRES LYAPEHYPLF RNRFMAWNSE |
| | | 121 | LEMAWRQYHA DEAIGAFYSN PRFVLIGDLV HGLMQRVFMH RLSPDAFSKG YDEMVRFVMG |
| | | 181 | GVKNV |
| 69 | WP_ | 1 | MPKATFFNLS KEKRKKIVEV LTEEFEKKNI QEATVKEIVQ KLGIARGSFY QYFETLEESY |
| | 192941430.1 | 61 | FYILELKTAD LHKSFMNLLV KYGDIYDVLE EYGEKIVEII FDKSCYNLYK NRYLLWNEKI |
| | | 121 | NRQWEEYKQK NMTNLIEVRN TDELLYISAL MHSLISRSYI EKWDKDKFLE KYKKYVRWIR |
| | | 181 | EGVRDE |
| 70 | WP_ | 1 | MPKNTFYNLP PEKREKIYEA LREEFNSNPF NKASVKDIVE NLNISRGSFY QYFEDLEESY |
| | 154536902.1 | 61 | FTILLKETTD IHSLFIEILK NENFDLCQSL EKFGDRISKL IFDERRYTLY KYRFLYWNED |
| | | 121 | LEKRWIEFYK KQGMECKHHS KNCIDNIGIN YEKINFIKAV IHNIIKRTFQ ENFSKETFLI |
| | | 181 | KYNQYINWII KGVD |
| 71 | WP_ | 1 | MPRSTFFSLK AEKRERIEKA LVSEFENRTL GDAKVKHVVE RLGISRGSFY QYFHNMQDAY |
| | 068368055.1 | 61 | FYILDLKLRF IHEGFFTLYR AEPQNIESVL EKYGVFLRES LYAPEHYPLF RNRFMAWNSE |
| | | 121 | LEMAWRQYHA DEAIGAFYSN PRFVLIGDLV HGLMQRVFMH RLSPDAFSKD YDEMVRFVMG |
| | | 181 | GVKNV |
| 72 | WP_ | 1 | MPKNTFNNLP PEKREKIYEA LREEFNSNPF NKASVKNIVE NLNISRGSFY QYFEDLEESY |
| | 091992952.1 | 61 | FTILLKETTD IHSLFIEILK DDNFDLCQSL EKFGNRISNL IFDERRYSLY KYRFLYWNED |
| | | 121 | LEKRWIEFYK KQAMECKHHS KNCIDNIDIN YEKVNFVKAV IHNIIKRAFQ ENFSKEAFLV |
| | | 181 | KYNQYVNWII KGVD |
| 73 | WP_ | 1 | MKKTFLNLSE EKKNTIIDAI KDEFLIHPFS DASVASIIDK AGISRGSFYQ YFENLEEAYF |
| | 005399124.1 | 61 | YILDLFTKDT HEIFFNLLKE NNFEYKKALE IYGNVLFEEM LQDEVYNLYK FKYLNWTQDL |
| | | 121 | EEKHTKYLVE NYGDEKKSSA LLSEREDMMF IKAVVHDLVC RLFSENWTKE EFLEKYNLYC |
| | | 181 | EWIIKGVNYG RTI |
| 74 | WP_ | 1 | MPKEVFFNLS DEKREKIIFV LKEEFKAKPF QKVNVKEIVE KAEIARGSFY QYFENLEDAY |
| | 009527141.1 | 61 | FTILEKETVD IHGLFMKIFL LKDKNLTEAL VEYGKEIADI IFDENAYMIY KNRYLYWNED |
| | | 121 | LNRSWESSHK HQEELFQDMS MDLEKIHFIK SIVHSLIERL FRESWNKEQF IEKYIKHLNW |
| | | 181 | IEKGVIYGNC |
| 75 | WP_ | 1 | MPKQTFYNLS EDKRNLIDEV LKDEFSTKPY KDCNVKNIVE RLSIARGSFY QYFDDLMDSY |
| | 002840625.1 | 61 | FYILDKETKD VHLLFMSVLK KNNNDVSVAL DAFGDEVCDV IFEDGEYNLY KFRYLYWNSE |
| | | 121 | LEDGWRATKK EYYEIFHDNS DCMKQELVYF MKAIVHSLIE RNFKENWSRD VFIEKYKLHI |
| | | 181 | NWLKKGVEHE DC |
| 76 | WP_ | 1 | MPKDTFYNLE KEKKDRITEV LTKEFSDKAF ADVNIKTIVE NLSIARGSFY QYFENLYDSY |
| | 099320921.1 | 61 | FYILDNKTYD IHLLFMQTLK KNNGDISLCL DDFGYEIADI IFDDSCYNLY KNRFLHWNEY |
| | | 121 | LEENRIKNHQ DYNKVFEKAK EMGVDLEKVN FFRAVVHALI QRNFREKWDK DLFIEKYKIH |
| | | 181 | IKWIKEGIFN EAR |
| 77 | WP_ | 1 | MPKDTFYNLE KEKKDRITEV LTKEFSDKAF ADVNIKTIVE NLSIARGSFY QYFENLYDSY |
| | 151409755.1 | 61 | FYILDNKTYD IHLLFMKTLK KNNGDISLCL DDFGYEIADI IFDDSCYNLY KNRFLHWNEY |
| | | 121 | LEENRIKNHQ DYNKVFDKAK EMGVDLEKVN FFRAVVHALI QRNFREKWDK DLFIEKYKIH |
| | | 181 | IKWIKEGIFN EAR |
| 78 | WP_ | 1 | MPKATFYNLS ADKQAKILAV LKTEFSTKPF NEITVKTIVD HLGMARGSFY QYFEDLEDSY |
| | 169792663.1 | 61 | FYVLDQETED IHLKFLECLT DQAGDLHEAL QNFGQAMSQF VFDQDKYDLY YYRYLHWTDA |
| | | 121 | LSRDWHQKKR SYYQSFYAKL NELDLDLEVM QYVKVVVHGL IERIFRDNLS QEQFLSIYQQ |
| | | 181 | QMEWIEKGIK E |
| 79 | WP_ | 1 | MPKATFYNLS ADKQAKILAV LKTEFSTKPF NEVTVKTIVD QLGMARGSFY QYFEDLEDSY |
| | 071409121.1 | 61 | FYVLDQETED IHLKFLECLT DQAGDLHEVL QNFGQAMSQF VFDQDKYDLY YYRYLHWTDA |
| | | 121 | LSRDWHQKKR SYYQSFYAKL NELDLDLEVM QYVKVVVHGL IERIFRDNLS QEQFLSIYQQ |
| | | 181 | QMEWIEKGIK E |
| 80 | WP_ | 1 | MPKQTFYNLS EDKKNLIDKV LKNEFSSKPF KDCNVKNIVD KLSIARGSFY QYFDDLKDSY |
| | 094209315.1 | 61 | FYILDKETKD VHLLFMSVLR QNNNDVLKAL DVFGDEVCDL IFEDGEYNLY KFRYLYWNSE |
| | | 121 | LEDGWKAKKK EYYEIFQNDT GGNKLELVCF MKATVHSLIE RNFKEGWSRD IFLQKYRLHI |
| | | 181 | NWLKEGIKNE DF |
| 81 | WP_ | 1 | MPKNTFNNLP KEKKEKIIRV LKEIFTAKNI QEVNVKEIVE KLNIARGSFY QYFDNLYDAY |
| | 180136139.1 | 61 | FLILELETED IHNVFMSIMR KNMFDIKKSL EEYGIRIADI LFKKESYSIY KYRYLYWDIE |
| | | 121 | LEKSWNKMNK INVVESEKMH FVKAVIHDLI ERMFKENWGK EKFINKYMEH LEMLKGGI |
| 82 | KXB66246.1 | 1 | MEIDFFFFEE YTFRKKVTQC HSQKEVIVPR STFFSLKAEK RERIEKALVS EFENRTLGDA |
| | | 61 | KVKHVVERLG ISRGSFYQYF HNMQDAYFYI LDLKLRFIHE GFFTLYRAEP QNIESVLEKY |
| | | 121 | GVFLRESLYA PEHYPLFRNR FMAWNSELEM AWRQYHADEA IGAFYSNPRF VLIGDLVHGL |
| | | 181 | MQRVFMHRLS PDAFSKDYDE MVRFVMGGVK NV |
| 83 | RRD94247.1 | 1 | MLVRTKRIER SLGSHKRKEE TMAKPTFYHL SEEKRQQIID VCKKEFEQNP IGQAKVSSIV |
| | | 61 | ETLGIARGSF YQYFEDIEEC YFTILKQETF ETHELFSQIL TRQPELFSAL ELYGQTLAEE |
| | | 121 | LFKPERYALY RNRYLFWTPV LDEKWKIFCG KTKALEGVNF PQLNTERLNL DNPAFIPELI |
| | | 181 | LYLKAIVHEL IRRLFVEKWT ADIFLKHYRL QMTWLKNGLC KN |
| 84 | WP_ | 1 | MPKNTFNNLP KEKKEKIIRV LKEIFTAKNI QEVNVKEIVE KLNIARGSFY QYFDNLYDAY |
| | 067320809.1 | 61 | FLILELETED IHNVFMSIMR KNMFDIKKSL EEYGIRIADI LFKKESYSIY KYRYLYWDIE |
| | | 121 | LEKSWNKMNK IDVVESEKMH FVKAVIHDLI ERMFKENWGK EKFINKYMEH LEMLKGGI |
| 85 | WP_ | 1 | MPKQTFYNLS EDKRNLIDEV LKDEFSTKPF KDCNVKNIVE RLSIARGSFY QYFDDLMDSY |
| | 094203142.1 | 61 | FYILDKETKD VHLLFMSVLK KNNNDVSRAL DAFGDEVCDV IFEDGEYNLY KFRYLYWNSE |
| | | 121 | LEDGWRTTKN EYYDIFHDNS DCMKQELVYF MKAIVHSLIE RNFKEEWSRD VFIEKYRLHI |
| | | 181 | NWLKKGVEHE DC |
| 86 | WP_ | 1 | MPKQTFYNLS EDKRNLIDEV LKDEFSTKPY KDCNVKNIVE RLSIARGSFY QYFDDLMDSY |
| | 002837851.1 | 61 | FYILDKETKD VHLLFMSVLK KNNNDVSVAL DAFGDEVCDV IFADGEYNLY KFRYLYWNSE |
| | | 121 | LEDGWRATKN EYYEIFHDNS DCMKQELVYF MKAIVHSLIE RNFKEEWSRD VFIEKYRLHI |
| | | 181 | NWLKKGVEHE DC |
| 87 | WP_ | 1 | MPKQTFYNLS EDKRNLIDEV LKDEFSTKPF KDCNVKNIVE RLSIARGSFY QYFDDLMDSY |
| | 094225300.1 | 61 | FYILDKETKD VHLLFMSVLK KNNNDVSRAL DVFGDEVCDL IFEDGEYNLY KFRYLYWNSE |
| | | 121 | LEDGWRATKN EYYDIFHDNS DCMKQELVYF MKAIIHSLIE RNFKEEWSRD VFIEKYRLHI |
| | | 181 | NWLKKGVEHE DC |
| 88 | WP_ | 1 | MPKQTFYNLS EDKRNLIDEV LKDEFSMKPY KDCNVKNIVE RLSIARGSFY QYFDDLMDSY |
| | 154274266.1 | 61 | FYILDKETKD VHLLFMSVLK KNNNDVPVAL DAFGDLLCDV IFEDGEYNLY KFRYLYWNSE |
| | | 121 | LEDGWRATKK EYYEIFHDDS DCMKQELVYF MKAIVHSLIE RNFKEEWTRD VFIEKYRLHI |
| | | 181 | NWLKKGVEHE DC |
| 89 | WP_ | 1 | MPKQTFYNLS EDKRNLIDEV LKDEFSTKPF KDCNVKNIVE RLSIARGSFY QYFDDLMDSY |
| | 094229311.1 | 61 | FYILDKETKD VHLLFMSVLK NNNNDVSVAL DAFGDEVCDL IFEDGEYNLY KFRYLYWNSE |
| | | 121 | LEDGWRATKN EYYEIFHDNS DCMKQEVVYF MKAIVHSLIE RNFKEEWSRD VFLEKYRLHI |
| | | 181 | NWLKKGVEHE DC |
| 90 | WP_ | 1 | MPKQTFYNLS EDKRNLIDEV LKDEFSTKPY KDCNVKNIVE RLSIARGSFY QYFDDLMDSY |
| | 060790933.1 | 61 | FYILDKETKD VHLLFMSVLK KNNNDVSVAL DVFGDEVCDV IFEDGEYNLY KFRYLYWNSE |
| | | 121 | LEDGWRATKN EYYEIFHDNS DCMKQELVYF MKAIIHSLIE RNFKEEWSRD VFIEKYRLHI |
| | | 181 | NWLKRGVEHE DC |
| 91 | WP_ | 1 | MPKATFYNLS ADKQAKILAV LKTEFSSKPF NEVTVKTIIE QLGMARGSFY QYFEDLEDSY |
| | 180764856.1 | 61 | FYVLDHETED IHLKFLECLT DQAGDLHEAL QNFGQAMSQF VFDQDKYDLY YYRYLHWTDA |
| | | 121 | LSRDWHEKKR SYYQSFYAKL NELDLDLEVM QYVKVVVHGL IERIFRDNLS QEQFLSIYQQ |
| | | 181 | QMEWIEKGIK E |
| 92 | OFK63194.1 | 1 | MWPKATFYN LSADKQ.AKIL AVLKTEFSTK PFNEVTVKTI VDQLGMARGS FYQYFEDLED |
| | | 61 | SYFYVLDQET EDIHLKFLEC LTDQAGDLHE VLQNFGQAMS QFVFDQDKYD LYYYRYLHWT |
| | | 121 | DALSRDWHQK KRSYYQSFYA KLNELDLDLE VMQYVKVVVH GLIERIFRDN LSQEQFLSIY |
| | | 181 | QQQMEWIEKG IKE |
| 93 | WP_ | 1 | MPKNTYFNLN DEKKKMVYDT LRCSFEKKSI RDITVKELVD ELKIPRGSFY QYFDSIEDAY |
| | 071026227.1 | 61 | FYVLEKEIVE IHDLFLNLLR DNNLNLISAL DLFANILADE IFKEKNYKLY RSRYISFDTD |
| | | 121 | LENKWKALRS KNPACQNPMF SLVDCEKLNF ISSVVHSLVK RLFNENWDRK DFLDHYKLYI |
| | | 181 | NWIKEGIR |
| 94 | WP_ | 1 | MPKDTFYNLD KEKKKKITDE LIYEFSDKPF SQVNIKTIVE NLGIARGSFY QYFNDLEDSY |
| | 117520944.1 | 61 | FYILDKKTHD IHILFMDTLK NNNGNIYDSL QEFGEDIAEI IFREDVYKLY KNRYLYWDEG |
| | | 121 | LNKSWENSHE NFRLVFNREQ SIGIDQEKIY FFRAVVHSLI ERNFREDWDK DTFVEKYKLH |
| | | 181 | IRWIMQGVIN GNR |
| 95 | WP_ | 1 | MPKQTFYNLS EDKKNLIDKV LKDEFSSKPF KDCNVKNIVD RLSIARGSFY QYFDDLKDSY |
| | 002835855.1 | 61 | FYILDKETKD VHLLFMSVLR QNNNDVLKAL DVFGDEVCDL IFEDGEYDLY KFRYLYWNSE |
| | | 121 | LEDSWKAKKK EYYEIFQNDT GGNKLELVYF MKATVHSLIE RNFKEGWSRD IFLQKYRLHI |
| | | 181 | NWLKEGIKNE DF |
| 96 | WP_ | 1 | MPKQTFYNLS EDKRNLIDEV LKDEFSTKPY KDCNVKNIVE RLSIARGSFY QYFDDLMDSY |
| | 035111990.1 | 61 | FYILDKETKD VHLLFMSVLK NNNNDVSEAL DAFGDEVCDV IFEDGEYNLY KFRYLYWNSE |
| | | 121 | LEDGWRATKN EYYEIFHDNS DCMRQELVYF MKAIVHSLIE RNFKEEWSRD VFIEKYRLHI |
| | | 181 | NWLKKGVEHE DC |
| 97 | WP_ | 1 | MPKETFYNLD QDKKSKITEV LISEFSEKPF EKVSVKAIVE RLGIARGSFY QYFEDLEDSY |
| | 073997938.1 | 61 | FYILDKKTHD MHMLFMTSLM KNDGDIITSL NDFGLNIADN IFKDECYNLY KNRFLFWNES |
| | | 121 | LNEDWINCHQ NYDKVVDKAD KMNVDLEKIY FFRAVVHSLV ERNFREEWDK VTFIEKYKIH |
| | | 181 | MDWIKKGIFN ANR |
| 98 | WP_ | 1 | MPKQTFYNLS EDKRNLIDEV LKDEFSTKPY KDCNVKNIVE RLSIARGSFY QYFDDLMDSY |
| | 012290153.1 | 61 | FYILDKETKD VHLLFRSVLK KNNNDVSVAL DAFGDEVCDV IFEDGEYNLY KFRYLYWNSE |
| | | 121 | LEDGWRATKN EYYDIFYDNS DCMKQELVYF MKAIVHSLIE RNFKEEWSRD VFIEKYRLHI |
| | | 181 | NWLKKGVGHE DC |
| 99 | WP_ | 1 | MPKQTFYNLS EDKKNLIDKV LKDEFSSKPF KDCNVKNIVD RLSIARGSFY QYFADLKDSY |
| | 110058607.1 | 61 | FYILDKETKD VHLLFMSVLR QNNNDVLKAL DVFGDEVCDL IFEDGEYNLY KFRYLYWNSE |
| | | 121 | LEDGWKATKK EYYEIFQNDT GGNKLELVCF MKATVHSLIE RNFKEGWSRD IFLQKYRLHI |
| | | 181 | NWLKEGIKNE DF |
| 100 | PKZ43199.1 | 1 | MVVPKATFYN LSADKQAKIL AVLKTEFSSK PFNEVTVKTI IEQLGMARGS FYQYFEDLED |
| | | 61 | SYFYVLDHET EDIHLKFLEC LTDQAGDLHE ALQNFGQAMS QFVFDQDKYD LYYYRYLHWT |
| | | 121 | DALSRDWHEK KRSYYQSFYA KLNELDLDLE VMQYVKVVVH GLIERIFRDN LSQEQFLSIY |
| | | 181 | QQQMEWIEKG IKE |

| | | |
|---|---|---|
| 101 | — | GATGACACAGTGTCAT |
| 102 | — | TGCTTGTGGGGGTAAGCGGTAGGTGAAATATTACAAATAGTATTTTTCGGTCATTTTAACTTGCTAT |
| | | TCTTGAAGAGGTTAGTACAATATGAATCGTGGATGACACAGTGTCATGCTTCAGATGACACAGTGT |
| | | CATGCAGTGTCATGCATGCTGATTCTTCAGCAAGACTACTACCTCATGAGAGTTATAGACTCATGG |
| | | ATCTTGCTTTGAAGGGTTTTGTACATTATAGGCTCCTATCACATGCTGAACCTATGGCCTATTACATT |
| | | TTTTTATATTTCAAGGAGGAAAAGACCAC |
| 103 | — | TGGCTTCAGCATGCTGATTCTTCAGCAAGACTACTACCTCATGAGAGTTATAGACTCATGGATCTTG |
| | | CTTTGAAGGGTTTTGTACATTATAGGCTCCTATCACATGCTGAACCTATGGCCTATTACATTTTTTTA |
| | | TATTTCAAGGAGGAAAAGACCAC |
| 104 | — | TG(GATGACACAGTGTCAT)GCTTCA(GATGACACAGTGTCATGCAT)GCTGATTCTTCAGCAAGACT |
| | | ACTACCTCATGAGAGTTATAGACTCATGGATCTTGCTTTGAAGGGTTTTGTACATTATAGGCTCCTA |
| | | TCACATGCTGAACCTATGGCCTATTACATTTTTTTATATTTCAAGGAGGAAAAGACCAC |
| 105 | — | CTTAAAATATAGTCATAGAATTAGGGCGTCGGTTTTAAAAGTGTGGTAAAATAATGGTCAAGATTA |
| | | (TGGATGACACAGTGTCATGCTTCAGATGACACAGTGTCATGCATGCTGATTCTTCAGCAAGACTAC |
| | | TACCTCATGAGAGTTATAGACTCATGGATCTTGCTTTGAAGGGTTTTGTACATTATAGGCTCCTATC |
| | | ACATGCTGAACCTATGGCCTATTACATTTTTTTATATTTCAAGGAGGAAAAGACCAC) |
| 106 | — | ATAAAACGAAAGGCTCAGTCGAAAGACTGGGCCTTTCGTTTTAT |
| 107 | — | [GGGS]n |
| 108 | — | Gn |
| 109 | — | [EAAAK]n |
| 110 | — | PA PAP |

Constitutive promoters in the first and second nucleic acids may be the same or different, and may be chosen based on the bacterial strain in which the nucleic acids will be utilized. For example, in any embodiment, the bacterial strain may be a lactic acid bacteria. Examples of suitable constitutive promoters from lactic acid bacteria include, but are not limited to, slpA from a Lactobacillus bacteria (e.g., acidophilus NCFM or rhamnosus), cplC from Lactobacillus fermentum BR11, ldh from Lactobacillus plantarum, Lactobacillus casei, and Lactobacillus reuteri, pgm from Lactobacillus agilis, ermB promoter from Enterococcus faecalis, and lacA from Lactobacillus lactis. A heme-responsive promoter comprising a constitutive promoter modified to include one or more hrtO repressor binding sites will be represented generically herein as “P_{n(hrtO)}”, where “n” is the constitutive promoter and “(hrtO)” represents modification with one or more hrtO repressor binding sites. For example, SEQ ID NO:102 represents heme-responsive promoter P_{slpA(hrtO)}, comprising the slpA promoter from either Lactobacillus acidophilus NCFM or Lactobacillus rhamnosus, modified to include one or more hrtO repressor binding sites from Lactobacillus lactis (SEQ ID NO:101). Similarly, P_{clpC(hrtO)} describes a heme-responsive promoter comprising the clpC promoter from Lactobacillus fermentum BR11, modified to include one or more hrtO repressor binding sites. P_{lacA(hrtO)} describes an hrtO-modified lacA promoter from Lactobacillus lactis. P_{ldh(hrtO)} describes an hrtO-modified ldh promoter from Lactobacillus plantarum, Lactobacillus casei, or Lactobacillus reuteri. P_{pgm(hrtO)} describes an hrtO-modified pgm promoter from Lactobacillus agilis.

The one or more hrtO repressor binding sites may be inserted into an slpA promoter, for example, of a Lactobacillus bacteria (e.g., Lactobacillus acidophilus NCFM or Lactobacillus rhamnosus), at any position within the promoter sequence sufficient to inhibit downstream transcription when HrtR is bound to the hrtO repressor binding site. One of skill in the art will be able to, without undue experimentation, determine a position of one or more of the hrtO repressor binding sites that allows for heme-responsive transcription.

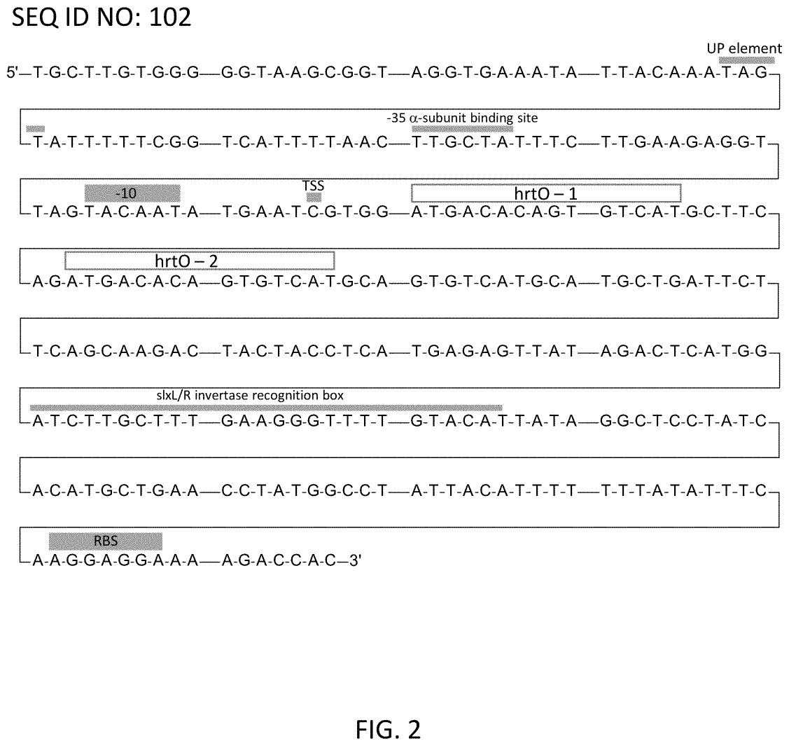

For example, one or more hrtO repressor binding sites may be inserted into the slpA promoter of Lactobacillus acidophilus NCFM as shown in FIG. 2, which depicts SEQ ID NO:102. In FIG. 2, the slpA promoter of Lactobacillus acidophilus NCFM is modified with two hrtO repressor binding sites (hrtO-1 and hrtO-2) from Lactobacillus lactis. Variations of SEQ ID NO:102 that retain the functionality of a heme-responsive promoter may also be used. For example, in any embodiment, a nucleic acid sequence that is at least 70% homologous, at least 80% homologous, at least 90% homologous, at least 95% homologous, or at least 98% homologous to SEQ ID NO:102 may be used.

In many embodiments, therefore, a heme-responsive repressor system may comprise a) a first nucleic acid comprising a heme-responsive promoter comprising an slpA promoter modified with one or more hrtO repressor binding sites and b) a second nucleic acid that encodes for the expression of an HrtR protein represented by any one of SEQ ID NOs:1-100 as shown in Table 1.

Heme-Responsive Promoters Using hrtO-Modified UTRs

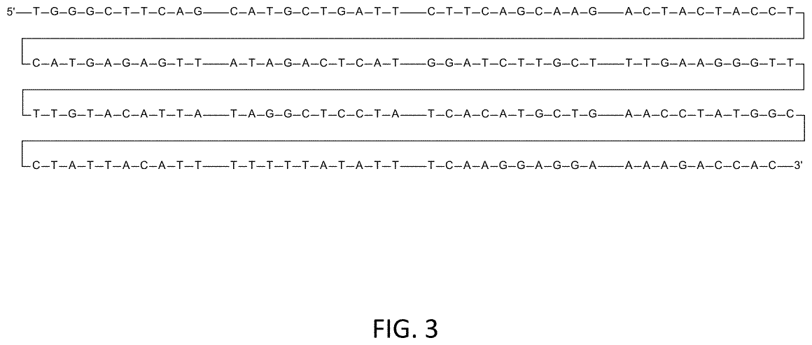

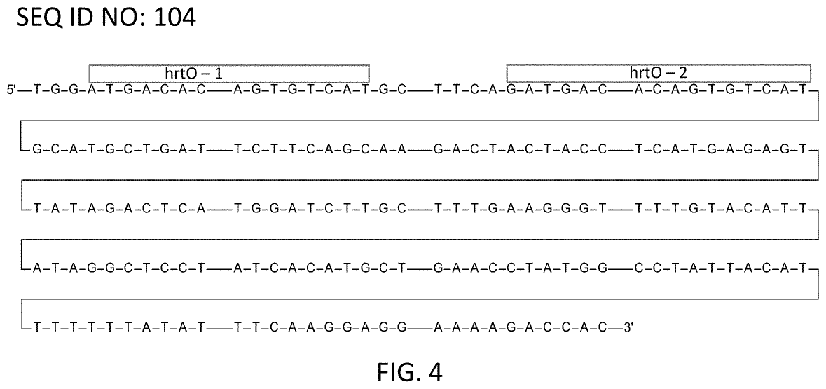

Some constitutive promoters comprise an untranslated region (UTR) which may be utilized for adapting the use of hrtO and HrtR as heme-responsive components of a promoter into any bacteria. For example, the slpA promoter of Lactobacillus acidophilus NFCM has a 5′ 190 base-pair (bp) UTR, which is shown in FIG. 3 and represented by SEQ ID NO:103. Other Lactobacillus bacteria, such as Lactobacillus rhamnosus also have an slpA promoter with a UTR that may also be used according to the various aspects and embodiments disclosed herein. One or more hrtO repressor binding sites may be inserted at any location in a UTR which is then fused to a constitutive promoter of any bacterial strain to generate a heme-responsive promoter. For example, in any embodiment, one or more hrtO repressor binding sites may be inserted at or close to the constitutive promoter transcription start site (TSS, e.g., just pass the +1 bp) that ends with the ribosome-binding site (RBS) needed for translation of the expressed gene transcript. For example, FIG. 3 depicts SEQ ID NO:103, the UTR of the slpA promoter from Lactobacillus acidophilus NFCM. FIG. 4 depicts SEQ ID NO:103 with an hrtO repressor binding site inserted 5 base pairs from the TSS. An additional hrtO binding site is inserted 6 bp downstream of the first hrtO binding site. While SEQ ID NO:103 represent a UTR with two hrtO repressor binding sites, a UTR may include any number of hrtO repressor binding sites, such as 1, 2, 3, 4, 5, 6, or more than 6. Additionally, in any embodiment, a variant of a UTR promoter may be used and modified with the one or more hrtO repressor binding sites. For example, one or more hrtO repressor binding sites may be inserted into a nucleotide sequence having at least 70%, 80%, 90%, or 95% homology to SEQ ID NO:103.

SEQ ID NO:103, as discussed above, illustrates one example where two hrtO repressor binding sites were inserted into the UTR of the slpA promoter of Lactobacillus acidophilus NCFM. However, the use of an slpA UTR is not limited to use within an slpA promoter. It has been discovered that, advantageously, insertion of the one or more hrtO repressor binding sites into a slpA UTR creates a nucleic acid sequence that can be fused to any constitutive promoter without a UTR or replace the UTR of a constitutive promoter having one. As such, an hrtO-modified UTR may be used in any bacterial strain to generate a heme-responsive promoter and repressor system. The bacterial strain may be a lactic acid bacteria other than Lactobacillus acidophilus NFCM, or any other bacteria. For example, FIG. 4 illustrates SEQ ID NO:105, whereby the 5′ UTR of the slpA promoter from Lactobacillus acidophilus NCFM modified with two hrtO repressor binding sites is fused to the clpC promoter of Lactobacillus fermentum BR11.

Other examples of suitable constitutive promoters include, but are not limited to, the lacA promoter from Lactobacillus lactis, the ldh promoter from Lactobacillus plantarum, Lactobacillus casei, or Lactobacillus reuteri, the ermB promoter from Enterococcus faecalis, or the pgm promoter from Lactobacillus agilis. Each may be fused to a UTR region of an slpA modified with one or more hrtO repressor binding sites from Lactobacillus acidophilus NCFM.

Additionally, the use of a UTR is not just limited to the UTR of an slpA promoter. A UTR of any bacteria may be modified with one or more hrtO repressor binding sites and fused to a constitutive promoter to generate a heme-responsive promoter.

Therefore, in yet another aspect, the present disclosure provides a heme-responsive repressor system comprising a) a first nucleic acid sequence comprising a constitutive promoter fused to a UTR of a bacterial promoter modified with one or more hrtO repressor binding sites and b) a second nucleic acid sequence comprising a constitutive promoter operably linked to a gene encoding an HrtR protein that binds to the one or more hrtO repressor binding sites. In any embodiment, the UTR may be a UTR from an slpA promoter region. In any embodiment, the UTR may be the 5′ UTR from the slpA from Lactobacillus acidophilus NFCM. Examples of suitable constitutive promoters to which an hrtO-modified UTR may be fused include, but are not limited to clpC, lacA, ldh, ermB, and pgm.

Heme-Sensing Bacteria

In any embodiment or aspect described above, a heme-responsive promoter may be operably linked to a target gene encoding a target protein or peptide. As such, any embodiment or aspect of a heme-responsive repressor system described above may be utilized to selectively express a protein or peptide in response to the presence of heme by including the heme-responsive promoter operably linked to the target gene. In many embodiments, the target gene is a reporter gene.

Therefore, in another aspect, the present disclosure provides a heme-responsive repressor system comprising a) first nucleic acid sequence comprising a heme-responsive promoter comprising one or more hrtO repressor binding sites and operably linked to a reporter gene; and b) a second nucleic acid sequence comprising any constitutive promoter operably linked to a gene encoding an HrtR protein that binds to the hrtO repressor binding site in the heme-responsive promoter. The heme-responsive promoter, as described herein above, comprises any constitutive promoter, such as a constitutive promoter of a lactic acid bacteria. Examples of such promoters include, but are not limited to, slpA, cplC, lacA, pgm, ermB, and ldh. In some embodiments, the constitutive promoter is fused to a nucleic acid sequence having at least 70%, 80%, 90%, or 95% homology to SEQ ID NO:104, which includes the one or more hrtO repressor binding sites, to generate the heme-responsive promoter which is operably linked to the reporter gene. In other embodiments, a nucleic acid sequence having at least 70%, 80%, 90%, or 95% homology to SEQ ID NO:105 is operably linked to the reporter gene.

The reporter gene may be any gene that, when expressed, produces a reporter peptide or protein. For example, the reporter gene may encode for green fluorescent protein (GFP), which is detectable by fluorimetry. Reporter proteins envisioned herein include fluorescent proteins, luminescent proteins, and enzymatic reporters, such as GusA or PepN. In any aspect, the methods described herein may make use of epitope tags and reporter gene sequences as detectable moieties. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-S-transferase (GST), horseradish peroxidase (HRP), chloramphenicol acetyltransferase (CAT) beta-galactosidase, betaglucuronidase, maltose binding protein, luciferase, green fluorescent protein (GFP), HcRed, DsRed, cyan fluorescent protein (CFP), yellow fluorescent protein (YFP), and auto fluorescent proteins including blue fluorescent protein (BFP).

A nucleic acid, as disclosed herein and in any embodiment, may comprise a terminator that marks the end of a gene during transcription. A terminator sequence mediates transcriptional termination by providing signals in the newly synthesized mRNA that trigger processes which release an mRNA from the transcriptional complex. Termination processes include the direct interaction of the mRNA secondary structure with the complex and/or the indirect activities of recruited termination factors. Release of the transcriptional complex frees RNA polymerase and related transcriptional machinery to begin transcription of new mRNAs. Terminator sequences include those known in the art and identified and described herein. Rho-independent, hairpin-forming terminator sequences are of particular use in lactic acid bacteria, and effective terminators include, but are not limited to, the rrnB T1 terminator sequence, represented by SEQ ID NO:106 (ATAAAACGAAAGGCTCAGTCGAAAGACTGGGCCTTTCGTTTTAT).

A heme-responsive repressor system, as described above, may be utilized to induce expression of a reporter gene in the presence of heme. Therefore, a bacteria genetically modified with a heme-responsive repressor system comprising a reporter gene, as described herein, can function as an in vivo biosensor for heme, producing a detectable reporter protein in the presence of heme. Therefore, in another aspect, the present disclosure provides an engineered bacterial cell genetically modified with a) first nucleic acid comprising a heme-responsive promoter comprising one or more hrtO repressor binding sites and operably linked to a reporter gene; and b) a second nucleic acid sequence encoding for the expression of an HrtR protein that binds to the hrtO repressor binding site in the heme-responsive promoter.

In one aspect, a heme-responsive promoter may comprise any constitutive promoter of the engineered bacterial cell and may comprise one or more hrtO repressor binding sites to which any one of the HrtR repressor proteins represented by SEQ ID NOs:1-100 in Table 1 may bind. The second nucleic acid may therefore encode the expression of an HrtR repressor proteins represented by SEQ ID NOs:1-100 in Table 1.

In another aspect, a constitutive promoter may include one or more hrtO repressor binding sites from a lactic acid bacteria, such as an hrtO repressor binding site from Lactobacillus lactis, as described above. The second nucleic acid may therefore encode the expression an HrtR repressor protein having at least 70%, at least 80%, at least 90%, or at least 95% homology to SEQ ID NOs:1-100. For example, the constitutive promoter of the engineered bacteria may be fused to a nucleic acid sequence having at least 70%, 80%, 90%, or 95% homology to SEQ ID NO:104 and the second nucleic acid encodes for an HrtR repressor protein having at least 70%, at least 80%, at least 90%, or at least 95% homology to SEQ ID NOs:1-100.

In many embodiments, the engineered bacterial cell is a gram-positive bacteria and a constitutive promoter of the gram-positive bacteria is fused to a nucleic acid sequence having at least 70%, 80%, 90%, or 95% homology to SEQ ID NO:104, which includes the one or more hrtO repressor binding sites, to generate the heme-responsive promoter which is operably linked to the reporter gene. The second nucleic acid may therefore encode the expression an HrtR repressor protein having at least 70%, at least 80%, at least 90%, or at least 95% homology to SEQ ID NOs:1-100.

Lactic acid bacteria are important industrial microbes in the dairy industry and are heavily targeted for use in vivo due to their FDA status as a generally regarded as safe (GRAS) probiotic organism. Therefore, in any embodiment, the engineered bacterial cell may be a lactic acid bacteria and the constitutive promoter of the lactic acid bacteria is fused to a nucleic acid sequence having at least 70%, 80%, 90%, or 95% homology to SEQ ID NO:104, which includes the one or more hrtO repressor binding sites, to generate the heme-responsive promoter which is operably linked to the reporter gene. The second nucleic acid may therefore encode the expression an HrtR repressor protein having at least 70%, at least 80%, at least 90%, or at least 95% homology to SEQ ID NOs:1-100.

In other embodiments, the engineered bacteria is Lactobacillus fermentum BR11 and a promoter having a nucleic acid sequence having at least 70%, 80%, 90%, or 95% homology to SEQ ID NO:105 is operably linked to the reporter gene. The second nucleic acid may therefore encode the expression an HrtR repressor protein having at least 70%, at least 80%, at least 90%, or at least 95% homology to SEQ ID NOs:1-100.

The reporter gene, in any of the above described aspects and embodiments, may encode for any reporter protein listed supra, for example, fluorescent proteins, luminescent proteins, bioluminescent proteins, enzyme, epitope tag, or the like.

Therapeutic Delivery Via Engineered Bacteria

In another aspect, a heme-responsive repressor system, as described herein, may comprise a heme-responsive promoter operably linked to a gene encoding a therapeutic protein or peptide (herein “therapeutic gene”). Therefore, in another aspect, the present disclosure provides a heme-responsive repressor system comprising a) first nucleic acid comprising a therapeutic gene operably linked to an upstream heme-responsive promoter comprising a constitutive promoter modified to include one or more hrtO repressor binding sites; and b) a second nucleic acid encoding for the expression of an HrtR protein that binds to the one or more hrtO repressor binding sites in the heme-responsive promoter. Upon heme-binding, the HrtR protein is unable to bind to the one or more hrtO repressor binding sites, therefore transcription of the downstream therapeutic gene commences, resulting in the generation of therapeutic protein or peptide.

Any bacteria may be genetically modified with the heme-responsive repressor system, as described above, to generate engineered bacteria. In one aspect, a heme-responsive promoter may comprise any constitutive promoter of the engineered bacteria, modified with one or more hrtO repressor binding sites native to the engineered bacteria and operably linked to a therapeutic gene. The second nucleic acid may therefore encode the expression of an HrtR repressor protein that binds to the native hrtO repressor binding site.

In another aspect, a heme-responsive promoter may comprise any constitutive promoter modified with one or more hrtO repressor binding site of a lactic acid bacteria, such as an hrtO repressor binding site from Lactobacillus lactis, as described above, which is operably linked to a therapeutic gene. The second nucleic acid may therefore encode the expression an HrtR repressor protein having at least 70%, at least 80%, at least 90%, or at least 95% homology to SEQ ID NOs:1-100. In one example, a constitutive promoter of the engineered bacteria may be fused to a nucleic acid sequence having at least 70%, 80%, 90%, or 95% homology to SEQ ID NO:104. The second nucleic acid of the heme-responsive repressor system encodes for an HrtR repressor protein having at least 70%, at least 80%, at least 90%, or at least 95% homology to SEQ ID NOs:1-100.

In many embodiments, the engineered bacterial cell is a gram-positive bacteria and, as such, is genetically modified with a heme-responsive repressor system comprising a first nucleic acid comprising a therapeutic gene operably linked to an upstream heme-responsive promoter comprising a constitutive promoter of the gram-positive bacteria fused to a nucleic acid sequence having at least 70%, 80%, 90%, or 95% homology to SEQ ID NO:104. The second nucleic acid of the heme-responsive repressor system may therefore encode the expression an HrtR repressor protein having at least 70%, at least 80%, at least 90%, or at least 95% homology to SEQ ID NOs:1-100.

Lactic acid bacteria are important industrial microbes in the dairy industry and are heavily targeted for use in vivo due to their FDA status as a generally regarded as safe (GRAS) probiotic organism. Therefore, in any embodiment, the engineered bacterial cell may be a lactic acid bacteria genetically modified with a heme-responsive repressor system comprising a first nucleic acid comprising a therapeutic gene operably linked to an upstream heme-responsive promoter comprising a constitutive promoter of the lactic acid bacteria fused to a nucleic acid sequence having at least 70%, 80%, 90%, or 95% homology to SEQ ID NO:104. The second nucleic acid of the heme-responsive repressor system may therefore encode the expression an HrtR repressor protein having at least 70%, at least 80%, at least 90%, or at least 95% homology to SEQ ID NOs:1-100.

In other embodiments, the engineered bacteria is Lactobacillus fermentum BR11 and a promoter having a nucleic acid sequence having at least 70%, 80%, 90%, or 95% homology to SEQ ID NO:105 is operably linked to the reporter gene. The second nucleic acid may therefore encode the expression an HrtR repressor protein having at least 70%, at least 80%, at least 90%, or at least 95% homology to SEQ ID NOs:1-100.

Examples of suitable therapeutic proteins include, but are not limited to, endogenous proteins, associated with probiotic function (e.g., LGG p40, spaC, SOD), antibodies (including IgG, IgE, IgA, IgM, scFv, and camlid antibodies—that target infectious agents, or host cell-surface proteins and antibody Fc), antimicrobial peptides of mammalian, viral, and bacterial origin (e.g., collistin, caerin, dermaseptin, LL-37, HBD-1/2/3, nisin, sakacin, lysozyme) antiviral peptides (e.g., HCV-CSA, Fuzeon), cytokines (e.g., IL-10), allergens (e.g., pollen, nut proteins), worm protein (e.g., hookworm protein), trefoil factor, dietary enzymes, mucin binding proteins (e.g., intJ, GroEL), invasins, antitoxins or any antigen derived from an infectious agent delivered as a vaccination target.

Optionally, and in any embodiment, a gene encoding a therapeutic peptide or protein may be conjoined, with or without a peptide linker (such as SEQ ID NO:107 [GGGS]n, SEQ ID NO:108 Gn, SEQ ID NO:109 [EAAAK]n, or SEQ ID NO:110 PAPAP where n is the number of motif repeats), to a reporter gene as described above (e.g., encoding a reporter protein), thereby yielding a fusion protein that, in the presence of heme (e.g., GI bleeding), produces a detectable reporter protein or peptide but also produces a therapeutic agent to treat the bleeding.

Engineered Lactic Acid Bacteria

Advantageously and surprisingly, it has been discovered that, despite the absence of a heme transporter (e.g., ChuA), lactic acid bacteria genetically engineered with the nucleic acids disclosed herein may selectively express a protein or peptide in response to a heme-rich environment. The ability of the lactic acid bacteria to function in a heme-rich environment is surprising since a) heme is known to be toxic to many bacteria, particularly those not having elaborate systems to maintain control over intracellular heme concentrations, and b) heme does not easily diffuse across cell membranes to be available for interaction with the heme-responsive promoter.

Without being bound by any theory, it is believed that the insertion of one or more hrtO repressor binding sites within a lactic acid promoter (for example, in the UTR of an slpA promoter) renders use of the lactic acid bacteria disclosed herein in the presence of heme (and iron) possible because lactic acid bacteria are gram-positive. Gram-positive bacteria are characterized by the presence of a single membrane layer (rather than two) and an acidic cell wall that can tolerate charged species such as iron. Lactic acid bacteria also possess ABC transporter proteins that share between 29% and 50% homology with the HrtAB heme efflux complex of L. lactis, which prevents accumulation of heme in those bacterial cell types. Therefore, though the engineered lactic acid bacteria do not possess a heme transporter, the presence of ABC transporter proteins may rendering the lactic acid bacteria more resistant to heme toxicity. Other types of gram-positive bacteria that may be used but are not lactic acid bacteria include, but are not limited to Lactovum, Xanthomonas, Erysipelotrichaceae, Bacillus, Clostridiales, Peptoniphilus, Peptostreptococcaceae, Ezakiella, Sneathia, Helococcus, Finegoldia, Anaerococcus, Leuconostoc, Pediococcus, Oenococcus, Bifidobacteria, Weisella, Tetragenoccus, Propionibacteria, Streptococcus, Sporolactobacillus, Carnobacteria, Vagococcus, Enterococcus, and Globicatella.

The various heme-responsive promoters and repressor systems are not, however, limited to use in just lactic acid bacteria or gram-positive bacteria. A heme-responsive repressor system may be developed based on the above disclosure for any bacteria, including gram-negative bacteria, but would include a third nucleic acid encoding for the co-expression of the ChuA membrane transporter.

Expression Vectors

An engineered lactic acid bacteria may be genetically modified with any of the heme-responsive repressor systems disclosed above using an expression vector. Therefore, in another aspect, the present disclosure provides an expression vector comprising a heme-responsive repressor system as defined above, wherein a heme-responsive promoter is operably linked to one or more of a reporter gene and a therapeutic gene. The expression vector may be any listed supra, for example, a plasmid, bacterial artificial chromosome, fosmid, cosmid, shuttle vector, a P1 vector, episome, or viral vector.

Methods of Genetically Modifying Bacteria

Any heme-responsive repressor system as disclosed herein supra may be introduced into a bacterial cell using any method known to those skilled in the art for such introduction. Such methods include transfection, transformation, transduction, infection (e.g., viral transduction), injection, microinjection, gene gun, nucleofection, nanoparticle bombardment, transformation, conjugation, by application of the nucleic acid in a gel, oil, or cream, by electroporation, using lipid-based transfection reagents, or by any other suitable transfection method. One of skill in the art will readily understand and adapt such methods using readily identifiable literature sources.

As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection (e.g., using commercially available reagents such as, for example, LIPOFECTIN® (Invitrogen Corp., San Diego, Calif.), LIPOFECTAMINE® (Invitrogen), FUGENE® (Roche Applied Science, Basel, Switzerland), JETPEI™ (Polyplus-transfection Inc., New York, N.Y.), EFFECTENE® (Qiagen, Valencia, Calif.), DREAMFECT™ (OZ Biosciences, France) and the like), or electroporation (e.g., in vivo electroporation). Suitable methods for transforming or transfecting host cells can be found in Sambrook, et al. (Molecular Cloning: A Laboratory Manual. 2nd, ed., Cold Spring harbor Laboratory, Cold Spring Harbor Laboratory Press, Cold Spring Harbor, N.Y., 1989), and other laboratory manuals.

Methods and materials of non-viral delivery of nucleic acids to cells further include biolistics, virosomes, liposomes, immunoliposomes, polycation or lipid-nucleic acid conjugates, naked DNA, artificial virions, and agent-enhanced uptake of DNA. Lipofection is described in U.S. Pat. Nos. 5,049,386, 4,946,787; and 4,897,355 and lipofection reagents are sold commercially (e.g., TRANSFECTAM™ and LIPOFECTIN™). Cationic and neutral lipids that are suitable for efficient receptor-recognition lipofection of polynucleotides include those disclosed in WO91/17424 and WO 91/16024.

Engineered bacteria may be grown for a desired time according to any known methods in the art, for example, in media and under environmental conditions suitable for bacterial growth. For example, bacteria may be grown in complex culture media including MRS, TGE, APT, BHI, TSB and TSBYE at a pH of about 7 to about 9 and a temperature of about 37° C. One of skill in the art will be familiar with suitable conditions and/or be able to optimize growth conditions for a particular bacterial strain.

Therefore, in another aspect, the present disclosure provides a method of genetically modifying a bacteria to generate an engineered bacteria comprising providing a vector comprising a heme-responsive repressor system as described above, inoculating the bacteria with the vector therefore generating the engineered bacteria; and maintaining the bacteria under conditions effective to promote the growth of the engineered bacteria. The bacteria may be any described above, for example, gram-positive bacteria or more particularly, lactic acid bacteria. Gram-negative bacteria may also be used, provided expression of the ChuA transporter is also induced.

Pharmaceutical Formulations

The engineered bacteria, as provided herein, genetically modified with a heme-responsive repressor system operably linked to one or more of a reporter gene or therapeutic gene, may be formulated in a composition for oral, topical, parenteral, or transdermal administration. These compositions may be in the form of pill, tablet, capsule, microcapsule, powder, sachet, dragee, gel, liquid, suspension, solution, food product, cream or granule, and may further comprise one or more pharmaceutically acceptable excipients, such as fillers, binders, lubricants, glidants, surfactants, pH adjusters, flavorings, colorings, antioxidants, buffers, and the like.

Exemplary pH adjusters include, but are not limited to, one or more of ammonium bicarbonate, ammonium carbonate, ammonium citrate, ammonium hydroxide, ammonium phosphate, calcium carbonate, calcium chloride, calcium citrate, calcium fumarate, calcium hydroxide, calcium phosphate, magnesium carbonate, magnesium citrate, magnesium hydroxide, magnesium phosphate, magnesium sulfate, potassium bicarbonate, potassium carbonate, potassium citrate, potassium fumarate, potassium hydroxide, potassium sulfate, sodium bicarbonate, sodium carbonate, sodium citrate, sodium fumarate, sodium hydroxide, and sodium phosphate.

Food products may include, but are not limited to, a dairy product, a yoghurt, an ice cream, a milk-based drink, a milk-based garnish, a pudding, a milkshake, an ice tea, a fruit or vegetable juice, a diet drink, a soda, a sports drink, jelly-like foods, a powdered drink mixture for dietary supplementation, an infant and baby food, a calcium-supplemented orange juice, a sauce or a soup.

Methods of Detecting, Diagnosing and/or Treating Bleeding

The engineered bacteria, as disclosed herein, maybe used to detect, diagnose, and/or treat bleeding in a mucosal environment of a subject. Therefore, in another aspect, the present disclosure provides a method of detecting bleeding in a mucosal environment of a subject comprising administering to a subject in need thereof a composition comprising an engineered bacteria genetically modified with a heme-responsive repressor system as described herein above, wherein the heme-responsive repressor system comprises a) first nucleic acid comprising a heme-responsive promoter comprising a constitutive promoter modified to include one or more hrtO repressor binding sites operably linked to a reporter gene; and b) a second nucleic acid comprising any constitutive promoter and encoding an HrtR protein that binds to the one or more hrtO repressor binding sites in the heme-responsive promoter of the first nucleic acid; and detecting the presence of the reporter protein in the subject. The reporter gene may be or encode for any reporter protein listed supra, for example, fluorescent proteins, luminescent proteins, bioluminescent proteins, enzyme, epitope tag, and the like. Expression of the reporter gene and therefore the presence of a reporter protein is proportionally to the amount of heme stimulus in the mucosal environment.

The presence of reporter protein may be detected and quantified by any known test, such as, but not limited to, polymerase chain reaction (PCR) analysis, nucleic acid tests, singleplex diagnostic tests, multiplex diagnostic tests, biomarker measurements and detections, imaging analysis and any combination thereof. Analysis of reporter protein levels may be performed in vivo, ex vivo, or in vitro.

In another aspect, the present disclosure provides a method of treating bleeding in a mucosal environment of a subject comprising administering to a subject in need thereof a composition comprising an engineered bacteria genetically modified with a) first nucleic acid comprising a heme-responsive promoter comprising a constitutive promoter modified to include one or more hrtO repressor binding sites operably linked to a reporter gene encoding a therapeutic protein; and b) a second nucleic acid comprising any constitutive promoter and encoding an HrtR protein that binds to the hrtO repressor binding site in the heme-responsive promoter of the first nucleic acid. The therapeutic gene may encode for any therapeutic protein, such as an endogenous protein associated with probiotic function (e.g., LGG p40, spaC), antibody (including IgG, IgE, IgA, scFv and camlid antibodies that target infectious agents, or host cell-surface protein and antibody Fc), an antimicrobial peptide of mammalian, viral, and bacterial origin (e.g., collistin, caerin, dermaseptin, LL-37, HBD-2), an antiviral peptide (e.g., HCV-CSA, Fuzeon), an cytokine (e.g., IL-10), an allergen (e.g., pollen, nut proteins), worm protein (e.g., hookworm protein), trefoil factor, a dietary enzyme, a mucin binding protein (e.g., intJ, GroEL), an invasin, an antitoxin, or any antigen derived from an infectious agent delivered as a vaccination target.

The mucosal environment may be the subject's gastrointestinal tract. The subject may be a mammal, such as an animal or a human subject. The subject may have or be at risk of developing a bleeding disease or disorder, such as, but not limited to, inflammatory bowel disease, colitis, peptic ulcer, gastritis, polyps, hemorrhoids, cirrhosis, infections, and cancers.

The composition may be administered to the subject orally, topically, parenterally, or transdermally, for example, as a pill, tablet, capsule, microcapsule, powder, sachet, dragee, gel, liquid, suspension, solution, cream or granule.

The sensors and methods provided herein present several advantages that make them suitable for detecting mucosal bleeding, and treating, managing or preventing a variety of conditions associated with mucosal bleeding in mammalian subjects, such as humans. In particular, the disclosed probiotic biosensors can drive expression of any gene of interest and exhibit gene expression proportionally to the amount of blood stimulus in the mucosal environment. Because of these properties, the biosensors provided herein can be fused with any biologic payload, and thus constitute live bio diagnostics and bio therapeutics.

### EXAMPLES

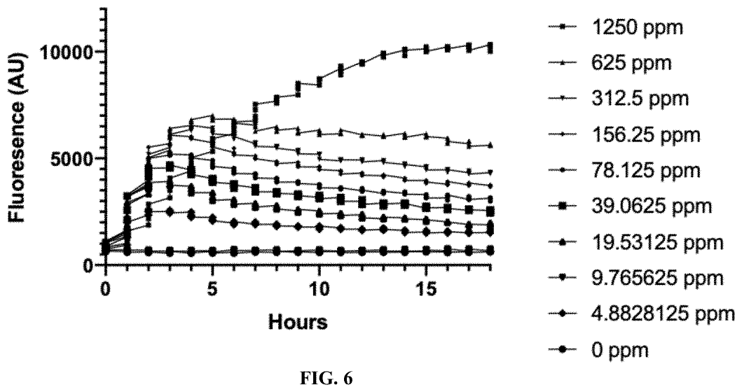

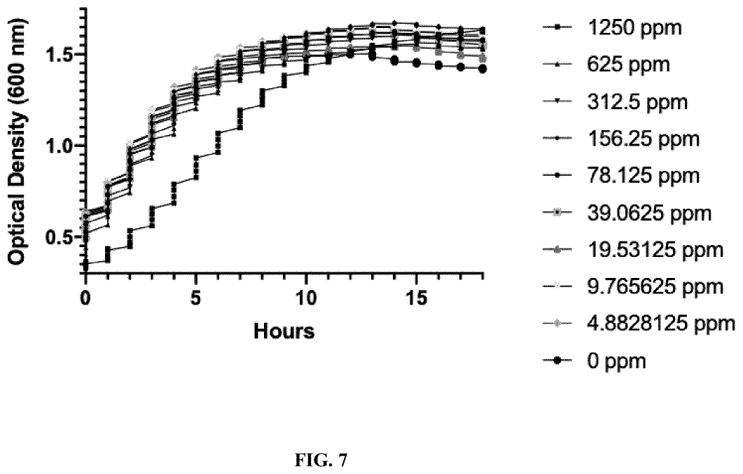

Example 1: In Vitro Response of a Genetically Modified Probiotic Bacterium to Heme. Strains of Lactobacillus rhamnosus GG were genetically modified with a vector comprising a first nucleic acid comprising the slpA promoter from Lactobacillus acidophilus NCFM modified to include two hrtO repressor binding sites from Lactobacillus lactis and operably linked to the fluorescence reporter mCherry gene, to drive the expression of the reporter gene in response to heme and a second nucleic acid comprising the slpA promoter operably linked to an HrtR protein, and a second nucleic acid. Engineered Lactobacillus rhamnosus GG were grown in culture media to which 0 ppm to 1250 ppm of heme was added. The fluorescence of the culture media (proportional to the concentration of mCherry therein) was analyzed over a 12 hour period. The results are shown in FIG. 6, demonstrating that higher the concentration of heme in the culture media, the stronger the expression of the reporter gene driven by the heme-responsive promoter. In FIG. 7, the data is normalized to bacterial growth to account for any variations in bacterial growth that may affect mCherry levels. These data demonstrate that the disclosed engineered bacteria are highly responsive to heme presence, and that the response of the engineered bacteria is proportional to the amount of heme to which they are exposed.

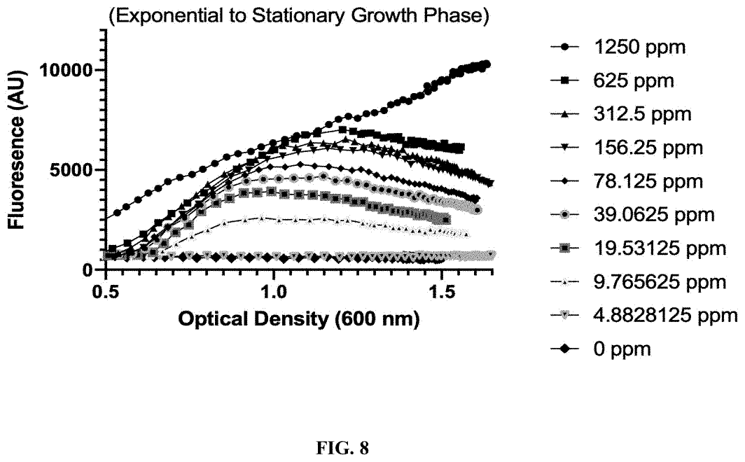

EXAMPLE 2: Bacterial Resistance to Heme Toxicity. The growth of the Lactobacillus rhamnosus GG from Example 1 was measured over time. The results are shown in FIG. 8 and illustrate that in all but the highest heme concentration (1250 ppm) the bacterial growth was similar to that of the same bacteria in absence of heme.

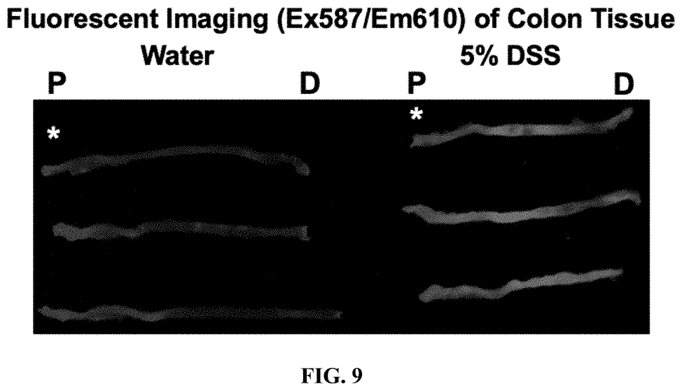

EXAMPLE 3: In Vivo Studies. Strains of Lactobacillus rhamnosus GG were genetically modified with a vector comprising a first nucleic acid comprising the slpA promoter form Lactobacillus acidophilus NCFM modified to include one or more hrtO repressor binding sites from Lactobacillus lactis and operably linked to the fluorescence reporter mCherry gene to drive the expression of the reporter gene in response to heme and a second nucleic acid comprising the slpA promoter operably linked to a gene encoding the HrtR repressor binding protein from Lactobacillus lactis. Lactobacillus rhamnosus GG were grown in culture media to an optical density of 0.9, prepared as doses of 10⁹ colony forming units (CFUs) in 300 μL of phosphate buffered saline (PBS), and administered via oral gavage to mice. Results are shown in FIG. 9. The mice were provided 5 days of normal drinking water (non-colitic/non-bleeding control group; left) or 5% DSS-treated drinking water (colitic/induced-bleeding experimental group; right). Gentian Violet used to identify proximal and distal orientation, which also emits a fluorescent signal that can be seen in the images in FIG. 8 (thus also providing a positive control signal for imaging). Proximal (P) and distal (D) orientation of organs are identified in the images. A higher signal is clearly seen in the DSS-treated colitic group, indicating induction of the engineered lactic acid bacteria administered to these mice, versus the control group where significantly less bleeding is expected. Visually, bleeding was confirmed grossly just prior to animal sacrifice and colon removal by observation of rectal bleeding only in the 5% DSS-treated mice.

1. A heme-responsive promoter sequence comprising a nucleic acid sequence that is at least 70% homologous to SEQ ID NO:104.

2. The heme-responsive promoter sequence of claim 1, wherein the heme-responsive promoter sequence is operably linked to a constitutive promoter of a lactic acid bacteria.

3. The heme-responsive promoter sequence of claim 2, wherein the constitutive promoter is selected from slpA, ldh, clpC, lacA, ermB or pgm.

4. The heme-responsive promoter sequence of claim 3, wherein the nucleic acid sequence is at least 70% homologous to SEQ ID NO:102.

5. The heme-responsive promoter sequence of claim 3, wherein the nucleic acid sequence is at least 70% homologous to SEQ ID NO:105.

6-18. (canceled)

19. A heme-responsive repressor system comprising:
a) a first nucleic acid comprising the heme-responsive promoter of claim 2; and
b) a second nucleic acid comprising a second constitutive promoter operably linked to a gene encoding an HrtR protein that binds to an hrtO repressor binding site in SEQ ID NO:104,
wherein the HrtR protein, when bound to heme, is not capable of binding to the hrtO repressor binding site, and
wherein the HrtR protein, when not bound to heme, is capable of binding to the hrtO repressor binding site.

20. The heme-responsive repressor system of claim 19, wherein the HrtR protein has an amino acid sequence that is at least 90% identical to a sequence selected from SEQ ID NOs: 1-100.

21. The heme-responsive repressor system of claim 19, wherein the first constitutive promoter and the second constitutive promoter are the same.

22. The heme-responsive repressor system of claim 21, wherein the first and second constitutive promoters are selected from slpA, ldh, clpC, lacA, ermB, or pgm.

23. The heme-responsive repressor system of claim 19, wherein the heme-responsive promoter is operably linked to a target gene.

24. The heme-responsive repressor system of claim 23, wherein the target gene is a reporter gene.

25. The heme-responsive repressor system of claim 24, wherein the reporter gene encodes for a fluorescent protein, a luminescent protein, a bioluminescent protein, an enzyme, or an epitope tag.

26. The heme-responsive repressor system of claim 23, wherein the target gene encodes expression of a therapeutic protein.

27. A bacterium comprising the heme-responsive repressor system of claim 19.

28. (canceled)

29. The bacterium of claim 27, wherein the bacterium is a Lactobacillus or Lactococcus bacteria.

30. A pharmaceutical composition comprising the bacteria of claim 27.

31. A method of detecting bleeding in a mucosal environment of a subject comprising:
a) administering to a subject in need thereof a composition comprising an engineered bacteria genetically modified with a heme-responsive repressor system, wherein the heme-responsive repressor system comprises:
a first nucleic acid comprising a heme-responsive promoter comprising having at least 70% homology to SEQ ID NO: 104 operably linked to a first constitutive promoter selected from slpA, ldh, clpC, lacA, ermB, or pgm; and
a second nucleic acid comprising a second constitutive promoter operably linked to a gene encoding the HrtR protein that binds to an hrtO repressor binding site in SEQ ID NO:104;
wherein the HrtR protein, when bound to heme, is not capable of binding to the hrtO repressor binding site, and
wherein the HrtR protein, when not bound to heme, is capable of binding to the hrtO repressor binding site; and
b) detecting the presence of the reporter protein in the subject.

32. A method of treating a gastric bleeding disorder in a mucosal gastrointestinal environment of a subject comprising:
a) administering to a subject in need thereof a composition comprising bacteria genetically modified with a heme-responsive repressor system, wherein the heme-responsive repressor system comprises:
a first nucleic acid comprising a heme-responsive promoter having at least 70% homology to SEQ ID NO: 104 operably linked to a first constitutive promoter selected from slpA, ldh, clpC, lacA, ermB, or pgm, operably linked to a therapeutic protein; and
a second nucleic acid comprising a second constitutive promoter operably linked to a gene encoding the HrtR protein that binds to an hrtO repressor binding site in SEQ ID NO: 104;
wherein the HrtT protein, when bound to heme, is not capable of binding to the hrtO repressor binding site, and
wherein the HrtR protein, when not bound to heme, is capable of binding to the hrtO repressor binding site; and
b) producing the therapeutic product in the subject's mucosal gastrointestinal environment in scale with the amount of heme present in the mucosal gastrointestinal environment.

33.-42. (canceled)

43. The method of claim 32, wherein the gastric bleeding disorder is inflammatory bowel disease, irritable bowel syndrome, colitis, peptic ulcer, gastritis, polyps, hemorrhoids, cirrhosis, an infection, or a cancer.

44. The heme-responsive promoter sequence of claim 1, wherein the nucleic acid sequence is SEQ ID NO:102, SEQ ID NO: 104, or SEQ ID NO: 105.


SEQUENCE LISTING (110 sequences)

SEQ ID NO: 1 - protein, 189 aa, Lactococcus lactis
MPKSTYFSLSDEKRKRVYDACLLEFQTHSFHEAKIMHIVKALDIPRGSFYQYFEDLKDSYYYILSQETVEIHDLFFNLLKEYPLEVALNKYKYLLLENLVNSPQYNLYKYRFLDWTYELERDWKPKGEVTVPARELDNPISQVLKSVIHNLVYRMFSENWDEQKFIETYDKEIKLLTEGLLNYVTESKK

SEQ ID NO: 2 - protein, 189 aa, Lactococcus lactis
MPKSTYFSLSDEKRKRVYDACLLEFQTHSFHEAKIMHIVKALDIPRGSFYQYFEDLKDSYYYILSQETVEIHDLFFNLLKEYPLEVSLNKYKYLLLENLVNSPQYNLYKYRFLDWTYELERDWKPKGEVTVPARELDNPISQVLKSVIHNLVYRMFSENWNEQKFIETYDKEIKLLTEGLLNYVTESKK

SEQ ID NO: 3 - protein, 189 aa, Lactococcus lactis
MPKSTYFSLSDEKRKRVYDACLLEFQTHSFHEAKIMHIVKALDIPRGSFYQYFEDLKDSYYYILSQETVEIHDLFFNLLKEYPLEVSLNKYKYLLLENLVNSPQYNLYKYRFLDWTYELERDWKPKGEVTVPTRELDNPISQVLKSVIHNLVYRMFSENWDEQKFIETYDKEIKLLTEGLLNYVTESKK

SEQ ID NO: 4 - protein, 189 aa, Lactococcus lactis
MPKSTYFSLSDEKRKRVYDACLLEFQTHSFHEAKIMHIVKALDIPRGSFYQYFEDLKDSYYYILSQETVEIHDLFFNLLKEYPLEVALDKYKYLLLENLVNSPQYNLYKYRFLDWTYELERDWKPKGEVTVPARELDNPISQVLKSVIHNLVYRMFSENWNEQKFIETYDKEIKLLTEGLLNYVTESKK

SEQ ID NO: 5 - protein, 194 aa, Lactococcus lactis subsp. cremoris
MIGEKMPKSTYFSLSDEKRKRVYDACLLEFQTHSFHEAKIMHIVKALDIPRGSFYQYFEDLKDSYYYILSQETVEIHDLFFNLLKEYPLEVALDKYKYLLLENLVNSPQYNLYKYRFLDWTYELERDWKPKGEVTVPARELDNPISQVLKSVIHNLVYRMFSENWNEQKFIETYDKEIKLLTEGLLNYVTESKK

SEQ ID NO: 6 - protein, 189 aa, Lactococcus lactis
MPKSTYFSLSDEKRKRVYDACLLEFQTHSFHEAKIMHIVKALDIPRGSFYQYFEDLKDSYYYILSQETVEIHDLFFNLLKEYPLEVALDKYKYLLLENLVNSPQYNLYKYRFLDWTYELERDWKPKGEVTVPARELDNPISQVLKSVIHNLVYRMFSENWNEQKFIETYDKEIKLLTEGLLNYVTENKK

SEQ ID NO: 7 - protein, 194 aa, Lactococcus lactis
MIGEKMPKSTYFSLSDEKRKRVYDACLLEFQTHSFHEAKIMHIVKALDIPRGSFYQYFEDLKDSYYYILSQETVEIHDLFFNLLKEYPLEVSLDKYKYLLLENLVNSPHYNLYKYRFLDWTYELERDWKPKGEVTVPARELDNPISQVLKSVIHNLVYRMFSENWNEQKFIETYDKEIKLLTEGLLNYVTESKK

SEQ ID NO: 8 - protein, 189 aa, Lactococcus lactis subsp. tructae
MPKSTYFSLSDEKRKRVYDACLLEFQTHSFHEAKIMHIVKALDIPRGSFYQYFEDLKDSYYYILSQETVEIHDLFFNLLKEYPLEVSLDKYKYLLLENLVNSPHYNLYKYRFLDWTYELERDWKPKGEVTVPARELDNPISQVLKSVIHNLVYRMFSENWNEQKFIETYDKEIKLLTEGLLNYVTESKK

SEQ ID NO: 9 - protein, 189 aa, Lactococcus lactis
MPKSTYFSLSDEKRNRVYDACLNEFQTHSFHEAKIMHIVKALDIPRGSFYQYFEDLKDAYFYVLSQETLEIHDLFFNLLKDYSIEEALDKYKYLLLENLIDSPQYQLYKYRFLDWTYELERDWKPKSSATVPASENNNPVSQVLKSVVHNLVYRLFSENWTEKTFIENYDKEIKLVTEGLLNYITDRKN

SEQ ID NO: 10 - protein, 189 aa, Lactococcus lactis
MPKSTYFSLSDEKRNRVYDACLNEFQTHSFHEAKIMHIVKALDIPRGSFYQYFEDLKDAYFYVLSQETLEIHDLFFNLLKDNSIEEALDKYKYLLLENLIDSPQYKLYKYRFLDWTYELERDWKPKSSATVPASENDNPISQVLKSVVHNLVYRLFSENWTEKTFIENYDKEIKLVTEGLLNYITDRKN

SEQ ID NO: 11 - protein, 189 aa, Lactococcus lactis
MPKSTYFSLSDEKRNRVYDACLNEFQTHSFHEAKIMHIVKALDIPRGSFYQYFEDLKDAYFYVLSQETLEIHDLFFNLLKDNSIEEALDKYKYLLLENLIDSPQYQLYKYRFLDWTYELERDWKPKSSATVPASENDNPVSQVLKSVVHNLVYRLFSENWTEKTFIENYDKEIKLVTEGLLNYITDRKN

SEQ ID NO: 12 - protein, 189 aa, Lactococcus lactis
MPKSTYFSLSDEKRNRVYDACLNEFQTHSFHEAKIMHIVKALDIPRGSFYQYFEDLKDAYFYVLSQETLEIHDLFFNLLKDNSIEEALDKYKYLLLENLIDSPQYQLYKYRFLDWTYELERDWKPQSSATVPASENDNPISQVLKSVVHNLVYRLFSENWTEKTFIENYDKEIKLVTEGLLNYITDRKN

SEQ ID NO: 13 - protein, 189 aa, Lactococcus lactis
MPKSTYFSLSDEKRNRVYDACLNEFQTHSFHEAKIMHIVKALDIPRGSFYQYFEDLKDAYFYVLSQETLEIHDLFFNLLKDNSIEEALDKYKYLLLENLIDSPQYQLYKYRFLDWTYELERDWKPQSSATVPASENDNPISQVLKSVVHNLVYRLFSENWTEKNFIENYDKEIKLVTEGLLNYITDRKN

SEQ ID NO: 14 - protein, 189 aa, Lactococcus lactis
MPKSTYFSLSDEKRNRVYDACLNEFQTHSFHEAKIMHIVKALDIPRGSFYQYFEDLKDAYFYVLSQETLEIHDLFFNLLKDNSIEEALDKYKYLLLENLIDSPQYKLYKYRFLDWTYELERDWKPQSSATVPASENDNPISQVLKSVVHNLVYRLFSENWTEKTFIENYDKEIKLVTEGLLNYITDRKN

SEQ ID NO: 15 - protein, 189 aa, Lactococcus lactis
MPKSTYFSLSDEKRNRVYDACLNEFQTHSFHEAKIMHIVKALDIPRGSFYQYFEDLKDAYFYVLSQETLEIHDLFFNLLKDNSVEESLDKYKYLLLENLIDSPQYQLYKYRFLDWTYELERDWKPKSSATVPASENDNPISQVLKSVVHNLVYRLFSENWTEKTFIENYDKEIKLVTEGLLNYITDRKN

SEQ ID NO: 16 - protein, 189 aa, Lactococcus lactis subsp. hordinae
MPKSTYFSLSDEKRNRVYDACLNEFQTHSFHEAKIMHIVKALDIPRGSFYQYFEDLKDAYFYVLSQETLEIHDLFFNLLKDNSIEEALDKYKYLLLENLIDSPQYQLYKYRFLDWTYDLERDWKPKSSATVPASENDNPVSQVLKSVVHNLVYRLFSENWTEKTFIENYDKEIKLVTEGLLNYITDRKN

SEQ ID NO: 17 - protein, 189 aa, Lactococcus lactis
MPKSTYFSLSDEKRNRVYDACLNEFQTHSFHEAKIMHIVKALDIPRGSFYQYFEDLKDAYFYVLSQETLEIHDLFFNLLKDNSIEEALDKYKYLLLENLIDSPQYQLYRYRFLDWTYELERDWKPQSSATVPASENDNPISQVLKSVVHNLVYRLFSENWTEKTFIENYDKEIKLVTEGLLNYITDRKN

SEQ ID NO: 18 - protein, 189 aa, Lactococcus lactis
MPKSTYFSLSDEKRNRVYDACLNEFQTHSFHEAKIMHIVKALDIPRGSFYQYFEDLKDAYFYVLSQETLEIHDLFFNLLKDNSIEEALDKYKYLLLENLIDSPQYQLYKYRFLDWTYELERDWKPKSSATVPASENNNPVSQVLKSVVHNLVYRLFSENWTEKTFIENYDKEIKLVTEGLLNYITDRKN

SEQ ID NO: 19 - protein, 189 aa, Lactococcus lactis
MPKSTYFSLSEEKRNRVYDACLNEFQTHSFHEAKIMHIVKALDIPRGSFYQYFEDLKDAYFYVLSQETLEIHDLFFNLLKDNSIEEALDKYKYLLLENLIDSPQYQLYKYRFLDWTYELERDWKPKSSATVPASENDNPVSQVLKSVVHNLVYRLFSENWTEKTFIENYDKEIKLVTEGLLNYITDRKN

SEQ ID NO: 20 - protein, 189 aa, Lactococcus lactis
MPKSTYFSLSDEKRNRVYDACLNEFQTHSFHEAKIMHIVKALDIPRGSFYQYFEDLKDAYFYVLSQETLEIHELFFNLLKDNSIEEALDKYKYLLLENLIDSPQYQLYKYRFLDWTYELERDWKPKSSATVPASENDNPVSQVLKSVVHNLVYRLFSENWTEKTFIENYDKEIKLVTEGLLNYITDRKN

SEQ ID NO: 21 - protein, 189 aa, Lactococcus lactis
MPKSTYFSLSDEKRNRVYDACLNEFQTHSFHEAKIMHIVKALDIPRGSFYQYFEDLKDAYFYVLSQETLEIHDLFFNLLKDNSIEESLDKYKYLLLENLIDSPQYKLYKYRFLDWTYELERDWKPQSSATVPASENDNPISQVLKSVVHNLVYRLFSENWTEKTFIENYDKEIKLVTEGLLNYITDRKN

SEQ ID NO: 22 - protein, 189 aa, Lactococcus lactis
MPKSTYFSLSDEKRNRVYDACLNEFQTHSFHESKIMHIVKALDIPRGSFYQYFEDLKDAYFYVLSQETLEIHDLFFNLLKDNSIEEALDKYKYLLLENLIDSPQYKLYKYRFLDWTYELERDWKPQSSATVPASENDNPISQVLKSVVHNLVYRLFSENWTEKTFIENYDKEIKLVTEGLLNYITDRKN

SEQ ID NO: 23 - protein, 189 aa, Lactococcus lactis
MPKSTYFSLSDEKRNRVYDACLNEFQTHSFHEAKIMHIVKALDIPRGSFYQYFEDLKDAYFYVLSQETLEIHDLFFNLLKDNSIEEALDKYKSLLLENLIDSPQYQLYKYRFLDWTYELERDWKPKSSATVPASENDNPVSQVLKSVVHNLVYRLFSENWTEKTFIENYDKEIKLVTEGLLNYITDRKN

SEQ ID NO: 24 - protein, 189 aa, Lactococcus lactis
MPKSTYFSLSDEKRNRVYDACLNEFQTHSFHEATIMHIVKALDIPRGSFYQYFEDLKDAYFYVLSQETLEIHDLFFNLLKDNSIEEALDKYKYLLLENLIDSPQYKLYKYRFLDWTYELERDWKPQSSATVPASENDNPISQVLKSVVHNLVYRLFSENWTEKTFIENYDKEIKLVTEGLLNYITDRKN

SEQ ID NO: 25 - protein, 189 aa, Lactococcus lactis
MPKSTYFSLSDEKRNRVYDACLNEFQTHSFHETKIMHIVKALDIPRGSFYQYFEDLKDAYFYVLSQETLEIHDLFFNLLKDNSIEEALDKYKYLLLENLIDSPQYKLYKYRFLDWTYELERDWKPQSSATVPASENDNPISQVLKSVVHNLVYRLFSENWTEKTFIENYDKEIKLVTEGLLNYITDRKN

SEQ ID NO: 26 - protein, 189 aa, Lactococcus lactis
MPKSTYFSLSDEKRNRVYDACLNEFQTHSFHEAKIMHIVKALDIPRGSFYQYFEDLKDAYFYVLSQETLEIHDLFFNLLKDNSIEEALDKYKYLLLENLIDSPQYQLYKYRFLDWTCELERDWKPKSSATVPASENDNPVSQVLKSVVHNLVYRLFSENWTEKTFIENYDKEIKLVTEGLLNYITDRKN

SEQ ID NO: 27 - protein, 189 aa, Lactococcus lactis
MPKSTYFSLSDEKRNRVYDACLNEFQTHSFHEAKIMHIVKALDIPRGSFYQYFEDLKDAYFYVLSQETLEIHELFFNLLKDNSIEEALDKYKYLLLENLIDSPQYQLYKYRFLDWTYELERDWKPQSSATVPASENDNPVSQVLKSVVHNLVYRLFSENWTEKTFIENYDKEIKLVTEGLLNYITDRKN

SEQ ID NO: 28 - protein, 189 aa, Lactococcus lactis
MPKSTYFSLSDEKRNRVYDACLNEFQTHSFHEAKIMHIVKALDIPRGSFYQYFEDLKDAYFYVLSQETLEIHDLFFNLLKDNSIEEALYKYKYLLLENLIDSPQYKLYKYRFLDWTYELERDWKPQSSATVPASENDNPISQVLKSVVHNLVYRLFSENWTEKTFIENYDKEIKLVTEGLLNYITDRKN

SEQ ID NO: 29 - protein, 189 aa, Lactococcus lactis
MPKSTYFSLSDEKRNRVYDACLNEFQTHSFHEAKIMHIVKALDIPRGSFYQYFEDLKDAYFYVLSQETLEIHDLFFNLLKDNSIEEALDKYKYLLLENLIDSPQYQLYKYRFLDWTYELERDWKSQSSATVPASENDNPISQVLKSVVHNLVYRLFSENWTEKNFIENYDKEIKLVTEGLLNYITDRKN

SEQ ID NO: 30 - protein, 187 aa, Lactococcus lactis
MPKSTYFSLSDEKRNRVYDACLNEFQTHSFHEAKIMHIVKALDIPRGSFYQYFEDLKDAYFYVLSQETLEIHDLFFNLLKDNSIEEALDKYKYLLLENLIDSPQYQLYKYRFLDWTYELERDWKPKSSATVPASENDNPVSQVLKSVVHNLVYRLFSENWTKKTFIENYDKEIKLVTEGLLNYITDR

SEQ ID NO: 31 - protein, 189 aa, Lactococcus lactis
MPKSTYFSLSDEKRNRVYDACLNEFQTHSFHEAKIMHIVKALDIPRGSFYQYFEDLKDAYFYVLSQETLEIHDLFFNLLKDNSIEEVLDKYKYLLLENLIDSPQYQLYKYRFLDWTYELERDWKPKSSATVPASENNNPVSQVLKSVVHNLVYRLFSENWTEKTFIENYDKEIKLVTEGLLNYITDRKN

SEQ ID NO: 32 - protein, 189 aa, Lactococcus lactis
MPKSTYFSLSDEKRNRVYDACLNEFQTHSFHEAKIMHIVKALDIPRGSFYQYFEDLKDAYFYVLSQETLEIHDLFFNLLKDNSIEEALDKYKYLLLENLIDSPQYQLYKCRFLDWTYELERDWKPQSSATVPASENDNPVSQVLKSVVHNLVYRLFSENWTEKTFIENYDKEIKLVTEGLLNYITDRKN

SEQ ID NO: 33 - protein, 189 aa, Lactococcus lactis
MPKSTYFSLSDEKRNRVYDACLNEFQTHSFHEAKIMHIVKALDIPRGSFYQYFEDLKDAYFYVLSQETLEIHDLFFNLLKYNSIEESLDKYKYLLLENLIDSPQYKLYKYRFLDWTYELERDWKPQSSATVPASENDNPISQVLKSVVHNLVYRLFSENWTEKTFIENYDKEIKLVTEGLLNYITDRKN

SEQ ID NO: 34 - protein, 189 aa, Lactococcus lactis
MPKSTYFSLSDEKRNRVYDACLNEFQTHSFHEAKIMHIVKALDIPRGSFYQYFEDLKDAYFYVLSQETLEIHELFFNLLIDNSIEEALDKYKYLLLENLIDSPQYQLYKYRFLDWTYELERDWKPKSSATVPASENDNPVSQVLKSVVHNLVYRLFSENWTEKTFIENYDKEIKLVTEGLLNYITDRKN

SEQ ID NO: 35 - protein, 189 aa, Lactococcus lactis
MPKSTYFSLSDEKCNRVYDACLNEFQTHSFHEAKIMHIVKALDIPRGSFYQYFEDLKDAYFYVLSQETLEIHDLFFNLLKDNSIEESLDKYKYLLLENLIDSPQYKLYKYRFLDWTYELERDWKPQSSATVPASENDNPISQVLKSVVHNLVYRLFSENWTEKTFIENYDKEIKLVTEGLLNYITDRKN

SEQ ID NO: 36 - protein, 189 aa, Lactococcus lactis
MPKSTYFSLSDEKRNRVYDACLNEFQTHSFHEAKIMHIVKALDIPRGSFYQYFEDLKDAYFYVLSKETLEIHDLFFNLLKYNSIEESLDKYKYLLLENLIDSPQYKLYKYRFLDWTYELERDWKPQSSATVPASENDNPISQVLKSVVHNLVYRLFSENWTEKTFIENYDKEIKLVTEGLLNYITDRKN

SEQ ID NO: 37 - protein, 187 aa, Lactococcus taiwanesis
MPKSTYFSLSDEKRNRVYDACLNEFQTHSFHEAKIMHIVKALDIPRGSFYQYFEDLKDAYFYVLSQETLEIHDLFFNLLKDNSIEEALDKYKYLLLENLIDSPQYQLYKYRFLDWTYELERDWKPKSSATVPASENDNPDSQVLKSVVHNLVYRLFSENWTKKTFIENYDKEIKLVTAGLLNYITDR

SEQ ID NO: 38 - protein, 154 aa, Lactococcus lactis subsp. lactis
MHIVKALDIPRGSFYQYFEDLKDAYFYVLSQETLEIHDLFFNLLKDNSIEESLDKYKYLLLENLIDSPQYKLYKYRFLDWTYELERDWKPQSSATVPASENDNPISQVLKSVVHNLVYRLFSENWTEKTFIENYDKEIKLVTEGLLNYITDRKN

SEQ ID NO: 39 - protein, 191 aa, Lactococcus fujiensis
MPKETYFSLPGEKRNRVYQACKLEFETHSYQDAKVMHVVKALNISRGSFYQYFEDLKDCYFFVLSNETIELHQLFIDLVKVYSIDEALEKYKFLLLEQLIRGENYEIYRHRFLDWNYDLDHAWKQKATGTFHSSKSNNPITQVLKSVVHDLVYRLFSENWDEETFIEHYEKEIEVVCIGLEAYYKNMKSHH

SEQ ID NO: 40 - protein, 195 aa, Lactococcus hircilactis
MPKETFFSLPDEKRMRVYQACKQEFEQHPFHEAKVMHIVKALGIPRGSFYQYFDDLMECYFYILSKETKELHGAFIDLIKVYPFEEAIDRYKYLLLEQLIHSDQYALYRYRFLSWDYRLDQAWAVRFGGTYHSEQKENPVIQVFKAVVHDLVYRLYAKAWDDQAFIAHYDRELSVVLNGVKHEMMGHPHEKRIKE

SEQ ID NO: 41 - protein, 190 aa, Lactococcus (unclassified)
MPKETYFSLPDEKRMRVYQACKQEFEKYSFHDAKIMHIVKALDIPRGSFYQYFEDLTDCYFYVLSKETTELHEIFMELIKHYSLDEALERYKYLLVDELLHGERASLYRFRFLDWNYELDQAWSERMVTSFEKERKNNATIQVFKAVVHDLIYQLYSKNWDATTFIAHYDSEISVLINGINTLTKVEKQE

SEQ ID NO: 42 - protein, 187 aa, Lactococcus petauri
MPKATYLKLPEEKKQRIYAACKTEFADKPFHQATVRNIVQSLEIPRGSFYQYFEDLTDCYIYVLSQETSELHQLFLDVIKKYPLEKALDEYKHLLLKELVHNENYAIYKYRFLDWNHELTLAWKKYNPQTFVSKEPVSPMISVFKAVVHDLVYRLFAEEWSDQEFVAHYDSEVAVIMNGLSGSQNHQ

SEQ ID NO: 43 - protein, 187 aa, Lactococcus garvieae
MPKATYLKLPEEKKQRIYAACKTEFADKPFHQATVRNIVQSLEIPRGSFYQYFEDLTDCYIYVLSQETSELHQLFLDAIKKYPLEKALDEYKHLLLQELVHNENYAIYKYRFLDWNHELTLAWKKYNPQTFVSKEPVSPMISVFKAVVHDLVYRLFAEEWSDQEFVAHYDSEVAVIMNGLSGSQNHQ

SEQ ID NO: 44 - protein, 374 aa, Lactococcus sp.
MPKATYLKLPEEKKQRIYAACKTEFADKPFHQATVRNIVQSLDIPRGSFYQYFEDLTDCYMYVLSQETRELHQLFLDAIQKYPLEQALDEYKHLLLKELVHNENYAIYKYRFLDWNHELTLAWKKYNPQTFVSKEPVSPMISVFKAVVHDLVYRLFAEEWSDQEFVAHYDSEVAVIMNGLSGSQNHQMPKATYLKLPEEKKQRIYAACKTEFADKPFHQATVRNIVQSLDIPRGSFYQYFEDLTDCYMYVLSQETRELHQLFLDAIQKYPLEQALDEYKHLLLKELVHNENYAIYKYRFLDWNHELTLAWKKYNPQTFVSKEPVSPMISVFKAVVHDLVYRLFAEEWSDQEFVAHYDSEVAVIMNGLSGSQNHQ

SEQ ID NO: 45 - protein, 187 aa, Lactococcus
MPKATYLKLPEEKKQRIYAACKTEFADKPFHQATVRNIVQSLEIPRGSFYQYFEDLTDCYIYVLSQETSELHQLFLDAIKKYPLEKALDEYKHLLLKELVHNENYAIYKYRFLDWNHELTLAWKKYNPQTFVSKEPVSPMISVFKAVVHDLVYRLFAEEWGDQEFVAHYDSEVAVIMKGLSGSQNHQ

SEQ ID NO: 46 - protein, 187 aa, Lactococcus
MPKATYLKLPEEKKQRIYAACKTEFADKPFHQATVRNIVQSLEIPRGSFYQYFEDLTDCYIYVLSQETSELHQLFLDAIKKYPLEKALDEYKHLLLKELVHNENYAIYKYRFLDWNHELTLAWKKYNPQTFVSKEPVSPMISVFKAVVHDLVYRLFAEEWSDQEFVAHYDSEVAVIMNGLSGSQNHQ

SEQ ID NO: 47 - protein, 187 aa, Lactococcus
MPKATYLKLPEEKKQRIYAACKTEFADKPFHQATVRNIVQSLEIPRGSFYQYFEDLTDCYIYVLSQETSELHQLFLEAIKKYPLEKALDEYKHLLLQELVHNENYAIYKYRFLDWNHELTLAWKKYNPQTFVSKEPVSPMISVFKAVVHDLVYRLFAEEWSDQEFVAHYDSEVAVIMNGLSGSQNHQ

SEQ ID NO: 48 - protein, 187 aa, Lactococcus garvieae
MPKATYLKLPEEKKQRIYAACKTEFADKPFHQATVRNIVQSLEIPRGSFYQYFEDLTDCYIYVLSQETSELHQLFLEAIKKYPLEKALDEYKHLLLKELVHNENYAIYKYRFLDWNHELTLAWKKYNPQTFVSKEPVSPMISVFKAVVHDLVYRLFAEEWSDQEFVAHYDSEVAVIMNGLSGSQNHQ

SEQ ID NO: 49 - protein, 187 aa, Lactococcus garvieae
MPKATYLKLPDEKKQRIYAACKTEFADKPFHQATVRNIVQSLDIPRGSFYQYFEDLTECYMYVLSKETSELHQLFLDAIQKYPLEQALDEYKHLLLKELVHNENYAIYKYRFLDWNHELTLAWKKYNPQTFVSKEPVSPMISVFKAVVHDLVYRLFAEEWSDQEFVAHYDSEVGVIMKGLSGSQNHQ

SEQ ID NO: 50 - protein, 187 aa, Lactococcus garvieae
MPKATYLKLPDEKKQRIYAACKTEFADKPFHQATVRNIVQSLDIPRGSFYQYFEDLTECYMYVLSKETSELHQLFLDAIQKYPLEQALDEYKHLLLKELVHNENYAFYKYRFLDWNHELSLAWKKYNPQTFVSKEPVSPMISVFKAVVHDLVYRLFAEEWSDQEFVAHYDSEVGVIMKGLSGSQNHQ

SEQ ID NO: 51 - protein, 187 aa, Lactococcus petauri
MPKATYLKLPEEKKQRIYAACKTEFADKPFHQATVRNIVQSLEIPRGSFYQYFEDLTDCYIYVLSQETSELHQLFLDAITKYPLEKALDEYKHLLLKELVHNENYAIYKYRFLDWNHELTLAWKKYNPQTFVSKEPVSPMISVFKAVVHDLVYRLFAEEWSDQEFVAHYDSEVAVIMNGLSGSQNHQ

SEQ ID NO: 52 - protein, 187 aa, Lactococcus
MPKATYLKLPEEKKQRIYAACKTEFADKPLHQATVRNIVQSLEIPRGSFYQYFEDLTDCYIYVLSQETSELHQLFLDAIKKYPLEKALDEYKHLLLKELVHNENYAIYKYRFLDWNHELTLAWKKYNPQTFVSKEPVSPMISVFKAVVHDLVYRLFAEEWSDQEFVAHYDSEVAVIMNGLSGSQNHQ

SEQ ID NO: 53 - protein, 187 aa, Lactococcus garvieae
MPKATYLKLPEEKKQRIYAACKTEFADKPFHQATVRNIVQSLDIPRGSFYQYFEDLTECYMYVLSKETSELHQLFLDAIQKYPLEQALDEYKHLLLKELVHNENYAIYKYRFLDWNHELTLAWKKYNPQTFVSKEPVSPMISVFKAVVHDLVYRLFAEEWSDQEFVAHYDSEVGVIMKGLSGSQNHQ

SEQ ID NO: 54 - protein, 187 aa, Lactococcus garvieae
MPKATYLKLPEEKKQRIYAACKTEFADKPFHQATVRNIVQSLDIPRGSFYQYFEDLTECYMYVLSKETSELHQLFLDAIQKYPLEQALDEYKHLLLKELVHNENYAIYKYRFLDWNHELTLAWKKYNPQTFVSKEPVSPMISVFKAVVHDLVYRLFAEEWSDQEFVAHYDSEVGVIMNGLSGSQNHQ

SEQ ID NO: 55 - protein, 187 aa, Lactococcus garvieae
MPKATYLKLPESKKQRIYAVCKKEFSDKPFHQATVRDIVQALEIPRGSFYQYFEDLTDCYMYVLSQETKELHNLFIDLIQKLPLMEALEEYKHLLLKELVHNENYSLYKYRFLDWNYELSLAWKKYNHQTFVTKEPVSPMISVFKSVVHDLVYRLFAEEWDDNEFITHYDNEIAIIMNGVSMSHIHH

SEQ ID NO: 56 - protein, 187 aa, Lactococcus garvieae; identical to SEQ ID NO: 55

SEQ ID NO: 57 - protein, 187 aa, Lactovum miscens
MPKETYYYLPDEKKTRVYLACRSEFETHSYHEAKIMHIVKALNIPRGSFYQYFDDLKDCYFYVLSKETIEIHDLFFELIKKHSLEESLEKYKYILLENLLHSSSYNLYRYRFLDWNYELEKDWKPKGTKTVSSVELNNPLIQVLKAVIHDIVHRLFSEKWSDEEFITVYNREIGLLKQGLTAYLSHG

SEQ ID NO: 58 - protein, 98 aa, Xanthomonas citri pv. citri
KYLVLENLVNSPQYNLYKYRFLDWTYELERDWKPKGEVTVPARELDNPISQVLKSVIHNLVYRMFSENWDEQKFIETYDKEIKLLTEGLLNYVSESKK

SEQ ID NO: 59 - protein, 192 aa, Erysipelotrichaceae bacterium OH741_COT-311
MPKETYFHLSKEKQQRIYEVCKQEFETHSFYEAKVSHIVKALGISRGSFYLYFEDLQDCYFYILQKETCELHDLFMQLMDHEDVVNALRKYQDVLLAHLLDPSVKRLYETRFLYWGHDLNQAWKAYQVTSSTFSPMPSDFIHYLKAVIHDLVFRLYSEGWDAETFKQHYQTVMHYVLYGISHRKEHAHEHHQ

SEQ ID NO: 60 - protein, 88 aa, Bacillus cereus
MPKSTYFSLSDEKRNRVYDACLNEFQTHSFHEAKIMHIVKALDIPRGSFYQYFEDLKDAYFYVLSQETLEIHDLFFNLLKDNSIEEAL

SEQ ID NO: 61 - protein, 210 aa, Clostridiales bacterium
MSLSGGVMPKKTFNNLSYERQQHVLAICRREFETHCFDAASVSAIVEQLGIARGTFYKYFEDLEDCYFYLLARETKEVHDIFFFLLKQNEFDIVTSLAQYGKRIAREIHDPQKYALYRNRYLGWTPLLQEKWQIYLEKLGKKAMPFADVVVEGYRAELLSGESIHLIKAVVHDLIARNFSENWTKAHFIKNYQRHIQLLTEGLKSNLLRD

SEQ ID NO: 62 - protein, 193 aa, Peptoniphilus ivorii
MPKATFYNLSEEKQARILEVLKHTFQTKTIFEANVKEIVEASEIARGSFYQYFENLEEAYFTILDRETVDIHDLFMALLTEQEGDVFAALEEYGDRLAEILFVEGNYGIYKNRFLYWTPGLEEGWQAYRKSAAAGASDRDRYLYREEMQFIKAVVHNLIQRNFLEKWDAQEFVRHYKQYAAWLKGGVHIDGTR

SEQ ID NO: 63 - protein, 193 aa, Peptostreptococcaceae bacterium oral taxon 113 str. W5053
MPKTTFHHLSEEKRQAVVAVLIHEFESYPLPDATVKHIVDALGIARGSFYQYFDSLTESYFYVLEREVIETHSLFLKLFHQCHRDTMQALEIYGEVLAKEIFQGKHYALYKNRYLYWNASLERDWRNYLAENQKDSSPMESMNENEPIRFVKAVIHALMQRLFMENWDRETFLKRYHQHIQWIKGGLTNVSPD

SEQ ID NO: 64 - protein, 188 aa, Ezakiella massiliensis
MPKNTFYNLPEEKKSRVVGVLKETFSAKSIYQANVKEIVEKLDIARGSFYKYFEDIEDAYFTILDMETADIHKVFLDFFKENDHDIHKALEAYGKALTEILFEPDTYGLYKNRYLNWTPDLEGKWRKYLKEFRPGEDDFSASDFDKDRLEFMKAAVHNLIQRTYLEAWDREEFLNHYNKYIAWLKGGI

SEQ ID NO: 65 - protein, 188 aa, Ezakiella peruensis
MPKNTFYNLPEEKKSRVVGILKETFSAKSIYQANVKEIVEKLDIARGSFYKYFEDIEDAYFTILDMETADIHKLFLSLLEKNNFDIHKALEAYGNALAEILFEPDTYGLYKNRYLNWTPDLEGKWRKYLKEFRPGEDDFSTSDFDRDRLEFMKAAVHNLIQRTYLEAWDREEFLNHYNKYIAWLKGGI

SEQ ID NO: 66 - protein, 186 aa, Sneathia amnii
MPKATFFNLSNEKRKKIVKVLTEEFEKKNIQEATVKEIVQKLGIARGSFYQYFETLEESYFYILELKTADLHKSFMNLLVKYGDIYDVLEEYGEKIVEIIFDKSCYNLYKNRYLLWNEKINRQWEEYKQKNMINLIEVRNTDELLYISALMHSLISRSYIEKWDKDKFLEKYKKYVRWIREGVRDE

SEQ ID NO: 67 - protein, 186 aa, Sneathia sp. DSM 16630; identical to SEQ ID NO: 66

SEQ ID NO: 68 - protein, 185 aa, Peptoniphilus coxii
MPRSTFFSLKAEKRERIEKALVSEFENRTLGDAKVKHVVERLGISRGSFYQYFHNMQDAYFYILDLKLRFIHEGFFTLYRAEPQNIESVLEKYGVFLRESLYAPEHYPLFRNRFMAWNSELEMAWRQYHADEAIGAFYSNPRFVLIGDLVHGLMQRVFMHRLSPDAFSKGYDEMVRFVMGGVKNV

SEQ ID NO: 69 - protein, 186 aa, Sneathia sp. DSM 16631
MPKATFFNLSKEKRKKIVEVLTEEFEKKNIQEATVKEIVQKLGIARGSFYQYFETLEESYFYILELKTADLHKSFMNLLVKYGDIYDVLEEYGEKIVEIIFDKSCYNLYKNRYLLWNEKINRQWEEYKQKNMTNLIEVRNTDELLYISALMHSLISRSYIEKWDKDKFLEKYKKYVRWIREGVRDE

SEQ ID NO: 70 - protein, 194 aa, Peptostreptococcus anaerobius
MPKNTFYNLPPEKREKIYEALREEFNSNPFNKASVKDIVENLNISRGSFYQYFEDLEESYFTILLKETTDIHSLFIEILKNENFDLCQSLEKFGDRISKLIFDERRYTLYKYRFLYWNEDLEKRWIEFYKKQGMECKHHSKNCIDNIGINYEKINFIKAVIHNIIKRTFQENFSKETFLIKYNQYINWIIKGVD

SEQ ID NO: 71 - protein, 185 aa, Peptoniphilus coxii
MPRSTFFSLKAEKRERIEKALVSEFENRTLGDAKVKHVVERLGISRGSFYQYFHNMQDAYFYILDLKLRFIHEGFFTLYRAEPQNIESVLEKYGVFLRESLYAPEHYPLFRNRFMAWNSELEMAWRQYHADEAIGAFYSNPRFVLIGDLVHGLMQRVFMHRLSPDAFSKDYDEMVRFVMGGVKNV

SEQ ID NO: 72 - protein, 194 aa, Peptostreptococcus sp. D1
MPKNTFNNLPPEKREKIYEALREEFNSNPFNKASVKNIVENLNISRGSFYQYFEDLEESYFTILLKETTDIHSLFIEILKDDNFDLCQSLEKFGNRISNLIFDERRYSLYKYRFLYWNEDLEKRWIEFYKKQAMECKHHSKNCIDNIDINYEKVNFVKAVIHNIIKRAFQENFSKEAFLVKYNQYVNWIIKGVD

SEQ ID NO: 73 - protein, 193 aa, Helcococcus kunzii
MKKTFLNLSEEKKNTIIDAIKDEFLIHPFSDASVASIIDKAGISRGSFYQYFENLEEAYFYILDLFTKDTHEIFFNLLKENNFEYKKALEIYGNVLFEEMLQDEVYNLYKFKYLNWTQDLEEKHTKYLVENYGDEKKSSALLSEREDMMFIKAVVHDLVCRLFSENWTKEEFLEKYNLYCEWIIKGVNYGRTI

SEQ ID NO: 74 - protein, 190 aa, Peptoanaerobacter stomatis
MPKEVFFNLSDEKREKIIFVLKEEFKAKPFQKVNVKEIVEKAEIARGSFYQYFENLEDAYFTILEKETVDIHGLFMKIFLLKDKNLTEALVEYGKEIADIIFDENAYMIYKNRYLYWNEDLNRSWESSHKHQEELFQDMSMDLEKIHFIKSIVHSLIERLFRESWNKEQFIEKYIKHLNWIEKGVIYGNC

SEQ ID NO: 75 - protein, 192 aa, Finegoldia magna
MPKQTFYNLSEDKRNLIDEVLKDEFSTKPYKDCNVKNIVERLSIARGSFYQYFDDLMDSYFYILDKETKDVHLLFMSVLKKNNNDVSVALDAFGDEVCDVIFEDGEYNLYKFRYLYWNSELEDGWRATKKEYYEIFHDNSDCMKQELVYFMKAIVHSLIERNFKENWSRDVFIEKYKLHINWLKKGVEHEDC

SEQ ID NO: 76 - protein, 193 aa, Anaerococcus sp. Marseille-P3625
MPKDTFYNLEKEKKDRITEVLTKEFSDKAFADVNIKTIVENLSIARGSFYQYFENLYDSYFYILDNKTYDIHLLFMQTLKKNNGDISLCLDDFGYEIADIIFDDSCYNLYKNRFLHWNEYLEENRIKNHQDYNKVFEKAKEMGVDLEKVNFFRAVVHALIQRNFREKWDKDLFIEKYKIHIKWIKEGIFNEAR

SEQ ID NO: 77 - protein, 193 aa, Anaerococcus sp. Marseille-P9784
MPKDTFYNLEKEKKDRITEVLTKEFSDKAFADVNIKTIVENLSIARGSFYQYFENLYDSYFYILDNKTYDIHLLFMKTLKKNNGDISLCLDDFGYEIADIIFDDSCYNLYKNRFLHWNEYLEENRIKNHQDYNKVFDKAKEMGVDLEKVNFFRAVVHALIQRNFREKWDKDLFIEKYKIHIKWIKEGIFNEAR

SEQ ID NO: 78 - protein, 191 aa, Globicatella sanguinis
MPKATFYNLSADKQAKILAVLKTEFSTKPFNEITVKTIVDHLGMARGSFYQYFEDLEDSYFYVLDQETEDIHLKFLECLTDQAGDLHEALQNFGQAMSQFVFDQDKYDLYYYRYLHWTDALSRDWHQKKRSYYQSFYAKLNELDLDLEVMQYVKVVVHGLIERIFRDNLSQEQFLSIYQQQMEWIEKGIKE

SEQ ID NO: 79 - protein, 191 aa, Globicatella sp. HMSC072A10
MPKATFYNLSADKQAKILAVLKTEFSTKPFNEVTVKTIVDQLGMARGSFYQYFEDLEDSYFYVLDQETEDIHLKFLECLTDQAGDLHEVLQNFGQAMSQFVFDQDKYDLYYYRYLHWTDALSRDWHQKKRSYYQSFYAKLNELDLDLEVMQYVKVVVHGLIERIFRDNLSQEQFLSIYQQQMEWIEKGIKE

SEQ ID NO: 80 - protein, 192 aa, Finegoldia magna
MPKQTFYNLSEDKKNLIDKVLKNEFSSKPFKDCNVKNIVDKLSIARGSFYQYFDDLKDSYFYILDKETKDVHLLFMSVLRQNNNDVLKALDVFGDEVCDLIFEDGEYNLYKFRYLYWNSELEDGWKAKKKEYYEIFQNDTGGNKLELVCFMKATVHSLIERNFKEGWSRDIFLQKYRLHINWLKEGIKNEDF

SEQ ID NO: 81 - protein, 178 aa, Streptobacillus felis
MPKNTFNNLPKEKKEKIIRVLKEIFTAKNIQEVNVKEIVEKLNIARGSFYQYFDNLYDAYFLILELETEDIHNVFMSIMRKNMFDIKKSLEEYGIRIADILFKKESYSIYKYRYLYWDIELEKSWNKMNKINVVESEKMHFVKAVIHDLIERMFKENWGKEKFINKYMEHLEMLKGGI

SEQ ID NO: 82 - protein, 212 aa, Peptoniphilus coxii
MEIDFFFFEEYTFRKKVTQCHSQKEVIVPRSTFFSLKAEKRERIEKALVSEFENRTLGDAKVKHVVERLGISRGSFYQYFHNMQDAYFYILDLKLRFIHEGFFTLYRAEPQNIESVLEKYGVFLRESLYAPEHYPLFRNRFMAWNSELEMAWRQYHADEAIGAFYSNPRFVLIGDLVHGLMQRVFMHRLSPDAFSKDYDEMVRFVMGGVKNV

SEQ ID NO: 83 - protein, 222 aa, Clostridiales bacterium COT073_COT-073
MLVRTKRIERSLGSHKRKEETMAKPTFYHLSEEKRQQIIDVCKKEFEQNPIGQAKVSSIVETLGIARGSFYQYFEDIEECYFTILKQETFETHELFSQILTRQPELFSALELYGQTLAEELFKPERYALYRNRYLFWTPVLDEKWKIFCGKTKALEGVNFPQLNTERLNLDNPAFIPELILYLKAIVHELIRRLFVEKWTADIFLKHYRLQMTWLKNGLCKN

SEQ ID NO: 84 - protein, 178 aa, Streptobacillus felis
MPKNTFNNLPKEKKEKIIRVLKEIFTAKNIQEVNVKEIVEKLNIARGSFYQYFDNLYDAYFLILELETEDIHNVFMSIMRKNMFDIKKSLEEYGIRIADILFKKESYSIYKYRYLYWDIELEKSWNKMNKIDVVESEKMHFVKAVIHDLIERMFKENWGKEKFINKYMEHLEMLKGGI

SEQ ID NO: 85 - protein, 192 aa, Finegoldia magna
MPKQTFYNLSEDKRNLIDEVLKDEFSTKPFKDCNVKNIVERLSIARGSFYQYFDDLMDSYFYILDKETKDVHLLFMSVLKKNNNDVSRALDAFGDEVCDVIFEDGEYNLYKFRYLYWNSELEDGWRTTKNEYYDIFHDNSDCMKQELVYFMKAIVHSLIERNFKEEWSRDVFIEKYRLHINWLKKGVEHEDC

SEQ ID NO: 86 - protein, 192 aa, Finegoldia magna
MPKQTFYNLSEDKRNLIDEVLKDEFSTKPYKDCNVKNIVERLSIARGSFYQYFDDLMDSYFYILDKETKDVHLLFMSVLKKNNNDVSVALDAFGDEVCDVIFADGEYNLYKFRYLYWNSELEDGWRATKNEYYEIFHDNSDCMKQELVYFMKAIVHSLIERNFKEEWSRDVFIEKYRLHINWLKKGVEHEDC

SEQ ID NO: 87 - protein, 192 aa, Finegoldia magna
MPKQTFYNLSEDKRNLIDEVLKDEFSTKPFKDCNVKNIVERLSIARGSFYQYFDDLMDSYFYILDKETKDVHLLFMSVLKKNNNDVSRALDVFGDEVCDLIFEDGEYNLYKFRYLYWNSELEDGWRATKNEYYDIFHDNSDCMKQELVYFMKAIIHSLIERNFKEEWSRDVFIEKYRLHINWLKKGVEHEDC

SEQ ID NO: 88 - protein, 192 aa, Finegoldia magna
MPKQTFYNLSEDKRNLIDEVLKDEFSMKPYKDCNVKNIVERLSIARGSFYQYFDDLMDSYFYILDKETKDVHLLFMSVLKKNNNDVPVALDAFGDLLCDVIFEDGEYNLYKFRYLYWNSELEDGWRATKKEYYEIFHDDSDCMKQELVYFMKAIVHSLIERNFKEEWTRDVFIEKYRLHINWLKKGVEHEDC

SEQ ID NO: 89 - protein, 192 aa, Finegoldia magna
MPKQTFYNLSEDKRNLIDEVLKDEFSTKPFKDCNVKNIVERLSIARGSFYQYFDDLMDSYFYILDKETKDVHLLFMSVLKNNNNDVSVALDAFGDEVCDLIFEDGEYNLYKFRYLYWNSELEDGWRATKNEYYEIFHDNSDCMKQEVVYFMKAIVHSLIERNFKEEWSRDVFLEKYRLHINWLKKGVEHEDC

SEQ ID NO: 90 - protein, 192 aa, Finegoldia magna
MPKQTFYNLSEDKRNLIDEVLKDEFSTKPYKDCNVKNIVERLSIARGSFYQYFDDLMDSYFYILDKETKDVHLLFMSVLKKNNNDVSVALDVFGDEVCDVIFEDGEYNLYKFRYLYWNSELEDGWRATKNEYYEIFHDNSDCMKQELVYFMKAIIHSLIERNFKEEWSRDVFIEKYRLHINWLKRGVEHEDC

SEQ ID NO: 91 - protein, 191 aa, Globicatella sanguinis
MPKATFYNLSADKQAKILAVLKTEFSSKPFNEVTVKTIIEQLGMARGSFYQYFEDLEDSYFYVLDHETEDIHLKFLECLTDQAGDLHEALQNFGQAMSQFVFDQDKYDLYYYRYLHWTDALSRDWHEKKRSYYQSFYAKLNELDLDLEVMQYVKVVVHGLIERIFRDNLSQEQFLSIYQQQMEWIEKGIKE

SEQ ID NO: 92 - protein, 193 aa, Globicatella sp. HMSC072A10
MVVPKATFYNLSADKQAKILAVLKTEFSTKPFNEVTVKTIVDQLGMARGSFYQYFEDLEDSYFYVLDQETEDIHLKFLECLTDQAGDLHEVLQNFGQAMSQFVFDQDKYDLYYYRYLHWTDALSRDWHQKKRSYYQSFYAKLNELDLDLEVMQYVKVVVHGLIERIFRDNLSQEQFLSIYQQQMEWIEKGIKE

SEQ ID NO: 93 - protein, 188 aa, Peptoniphilus raoultii
MPKNTYFNLNDEKKKMVYDTLRCSFEKKSIRDITVKELVDELKIPRGSFYQYFDSIEDAYFYVLEKEIVEIHDLFLNLLRDNNLNLISALDLFANILADEIFKEKNYKLYRSRYISFDTDLENKWKALRSKNPACQNPMFSLVDCEKLNFISSVVHSLVKRLFNENWDRKDFLDHYKLYINWIKEGIR

SEQ ID NO: 94 - protein, 193 aa, Anaerococcus nagyae
MPKDTFYNLDKEKKKKITDELIYEFSDKPFSQVNIKTIVENLGIARGSFYQYFNDLEDSYFYILDKKTHDIHILFMDTLKNNNGNIYDSLQEFGEDIAEIIFREDVYKLYKNRYLYWDEGLNKSWENSHENFRLVFNREQSIGIDQEKIYFFRAVVHSLIERNFREDWDKDTFVEKYKLHIRWIMQGVINGNR

SEQ ID NO: 95 - protein, 192 aa, Finegoldia magna
MPKQTFYNLSEDKKNLIDKVLKDEFSSKPFKDCNVKNIVDRLSIARGSFYQYFDDLKDSYFYILDKETKDVHLLFMSVLRQNNNDVLKALDVFGDEVCDLIFEDGEYDLYKFRYLYWNSELEDSWKAKKKEYYEIFQNDTGGNKLELVYFMKATVHSLIERNFKEGWSRDIFLQKYRLHINWLKEGIKNEDF

SEQ ID NO: 96 - protein, 192 aa, Finegoldia magna
MPKQTFYNLSEDKRNLIDEVLKDEFSTKPYKDCNVKNIVERLSIARGSFYQYFDDLMDSYFYILDKETKDVHLLFMSVLKNNNNDVSEALDAFGDEVCDVIFEDGEYNLYKFRYLYWNSELEDGWRATKNEYYEIFHDNSDCMRQELVYFMKAIVHSLIERNFKEEWSRDVFIEKYRLHINWLKKGVEHEDC

SEQ ID NO: 97 - protein, 193 aa, Anaerococcus urinomassiliensis
MPKETFYNLDQDKKSKITEVLISEFSEKPFEKVSVKAIVERLGIARGSFYQYFEDLEDSYFYILDKKTHDMHMLFMTSLMKNDGDIITSLNDFGLNIADNIFKDECYNLYKNRFLFWNESLNEDWINCHQNYDKVVDKADKMNVDLEKIYFFRAVVHSLVERNFREEWDKVTFIEKYKIHMDWIKKGIFNANR

SEQ ID NO: 98 - protein, 192 aa, Finegoldia magna
MPKQTFYNLSEDKRNLIDEVLKDEFSTKPYKDCNVKNIVERLSIARGSFYQYFDDLMDSYFYILDKETKDVHLLFRSVLKKNNNDVSVALDAFGDEVCDVIFEDGEYNLYKFRYLYWNSELEDGWRATKNEYYDIFYDNSDCMKQELVYFMKAIVHSLIERNFKEEWSRDVFIEKYRLHINWLKKGVGHEDC

SEQ ID NO: 99 - protein, 192 aa, Finegoldia magna
MPKQTFYNLSEDKKNLIDKVLKDEFSSKPFKDCNVKNIVDRLSIARGSFYQYFADLKDSYFYILDKETKDVHLLFMSVLRQNNNDVLKALDVFGDEVCDLIFEDGEYNLYKFRYLYWNSELEDGWKATKKEYYEIFQNDTGGNKLELVCFMKATVHSLIERNFKEGWSRDIFLQKYRLHINWLKEGIKNEDF

SEQ ID NO: 100 - protein, 193 aa, Globicatella sanguinis
MVVPKATFYNLSADKQAKILAVLKTEFSSKPFNEVTVKTIIEQLGMARGSFYQYFEDLEDSYFYVLDHETEDIHLKFLECLTDQAGDLHEALQNFGQAMSQFVFDQDKYDLYYYRYLHWTDALSRDWHEKKRSYYQSFYAKLNELDLDLEVMQYVKVVVHGLIERIFRDNLSQEQFLSIYQQQMEWIEKGIKE

SEQ ID NO: 101 - DNA, 16 bp, Lactobacillus lactis
gatgacacagtgtcat

SEQ ID NO: 102 - DNA, 296 bp, Artificial Sequence
  Synthetic
tgcttgtgggggtaagcggtaggtgaaatattacaaatagtatttttcggtcattttaacttgctattcttgaagaggttagtacaatatgaatcgtggatgacacagtgtcatgcttcagatgacacagtgtcatgcagtgtcatgcatgctgattcttcagcaagactactacctcatgagagttatagactcatggatcttgctttgaagggttttgtacattataggctcctatcacatgctgaacctatggcctattacatttttttatatttcaaggaggaaaagaccac

SEQ ID NO: 103 - DNA, 158 bp, Lactobacillus acidophilus
tggcttcagcatgctgattcttcagcaagactactacctcatgagagttatagactcatggatcttgctttgaagggttttgtacattataggctcctatcacatgctgaacctatggcctattacatttttttatatttcaaggaggaaaagaccac

SEQ ID NO: 104 - DNA, 190 bp, Artificial Sequence
  Synthetic
tggatgacacagtgtcatgcttcagatgacacagtgtcatgcatgctgattcttcagcaagactactacctcatgagagttatagactcatggatcttgctttgaagggttttgtacattataggctcctatcacatgctgaacctatggcctattacatttttttatatttcaaggaggaaaagaccac

SEQ ID NO: 105 - DNA, 256 bp, Artificial Sequence
  Synthetic
cttaaaatatagtcatagaattagggcgtcggttttaaaagtgtggtaaaataatggtcaagattatggatgacacagtgtcatgcttcagatgacacagtgtcatgcatgctgattcttcagcaagactactacctcatgagagttatagactcatggatcttgctttgaagggttttgtacattataggctcctatcacatgctgaacctatggcctattacatttttttatatttcaaggaggaaaagaccac

SEQ ID NO: 106 - DNA, 44 bp, Artificial Sequence
  Synthetic
ataaaacgaaaggctcagtcgaaagactgggcctttcgttttat

SEQ ID NO: 107 - protein, 4 aa, Artificial Sequence
  Synthetic
  REPEAT (1)..(4)
GGGS

SEQ ID NO: 108 - protein, 1 aa, Artificial Sequence
  Synthetic
  Repeat (1)..(1)
G

SEQ ID NO: 109 - protein, 5 aa, Artificial Sequence
  Synthetic
  REPEAT (1)..(5)
EAAAK

SEQ ID NO: 110 - protein, 5 aa, Artificial Sequence
  Synthetic
PAPAP